(19) United States
(12) Patent Application Publication
(10) Pub. No.: US 2025/0364865 A1
(43) Pub. Date: Nov. 27, 2025

(54) MOTOR ASSEMBLY FOR DRUG DELIVERY DEVICE

(71) Applicant: Regeneron Pharmaceuticals, Inc., Tarrytown, NY (US)

(72) Inventors: Bryan C. GRYGUS, Clifton Park, NY (US); Trevor LANGLEY, Rensselaer, NY (US); Daniel HALBIG, Ballston Lake, NY (US); Jessica MIKLINSKI, Cohoes, NY (US); Alexander ROUMANIDAKIS, Schenectady, NY (US); Igor DROBNJAK, Halfmoon, NY (US); Katriona BLEZY, Delmar, NY (US)

(73) Assignee: Regeneron Pharmaceuticals, Inc., Tarrytown, NY (US)

(21) Appl. No.: 19/214,582

(22) Filed: May 21, 2025

Related U.S. Application Data
(60) Provisional application No. 63/651,741, filed on May 24, 2024.

(51) Int. Cl. H02K 5/22; A61M 5/20; H02K 7/116; H02K 11/33

(52) U.S. Cl. CPC H02K 5/225; A61M 5/20; H02K 7/116; H02K 11/33; A61M 2005/206

(57) ABSTRACT

A motor assembly described herein generally includes a support structure having a first side opposite a second side; a motor mount extending from the first side of the support structure, the motor mount including: a first side opposite a second side; and a first raised portion adjacent a second raised portion; a motor retained by the motor mount; and a first connector and a second connector mechanically securing the motor to the motor mount and configured to form an electrical connection between the motor and a controller, wherein the first connector and the second connector extend from the first side of the motor mount, over the motor mount, to the second side of the motor mount, wherein the first connector is at least partially positioned between the first raised portion and the second raised portion of the motor mount.

### CROSS-REFERENCE TO RELATED APPLICATION(S)

This patent application claims priority to U.S. Provisional Patent Application No. 63/651,741 filed on May 24, 2024, the entirety of which is incorporated herein by reference.

### TECHNICAL FIELD

Various aspects of the present disclosure relate generally to motor assemblies. In particular, aspects of the present disclosure relate to motor assemblies for drug delivery devices, mechanical and/or electrical connectors for the motor assemblies, and methods associated with the motor assemblies.

### INTRODUCTION

Pharmaceutical therapies are often used for treating disease in patients, and such therapies may be delivered to patients in a variety of ways, e.g., through auto-injectors and/or through electromechanical injection devices. It is often desirable to utilize pharmaceutical therapy delivery devices with structural stability that endures over the lifetime of the devices. Further, it may be desirable for a drug delivery system to be designed with components that individually perform multiple functions, so as to reduce the overall size and complexity of the drug delivery system. Therefore, devices for delivering drug therapies with reduced complexity and improved functionality, and methods made possible by such instruments, are highly sought after to provide improved outcomes for users and manufacturers.

### SUMMARY OF THE DISCLOSURE

According to an aspect of the disclosure, the present disclosure is directed to a motor assembly, e.g., for an injection device. The motor assembly may include: a support structure having a first side opposite a second side; a motor mount extending from the first side of the support structure, the motor mount including: a first side opposite a second side; and a first raised portion adjacent a second raised portion; a motor retained by the motor mount; and a first connector and a second connector mechanically securing the motor to the motor mount and configured to form an electrical connection between the motor and a controller, wherein the first connector and the second connector extend from the first side of the motor mount, over the motor mount, to the second side of the motor mount, wherein the first connector is at least partially positioned between the first raised portion and the second raised portion of the motor mount.

In embodiments, the motor mount includes a linear portion and a curved portion, and wherein the first connector and the second connector are at least partially positioned on the linear portion. In embodiments, the motor mount further includes a third raised portion, and wherein the second connector is at least partially positioned between the second raised portion and the third raised portion of the motor mount. In embodiments, the motor mount further comprises: a first post extending from the first side of the motor mount and disposed between the first raised portion and the second raised portion of the motor mount, and a second post extending from the first side of the motor mount and disposed between the second raised portion and the third raised portion of the motor mount, wherein the first post extends through the first connector and the second post extends through the second connector. In embodiments, the first connector is configured to electrically couple a first terminal of the motor to a first terminal of a controller, and wherein the second connector is configured to electrically couple a second terminal of the motor to a second terminal of the controller.

According to an aspect of the disclosure, the motor assembly may include: a support structure including: a first side opposite a second side; an opening defined through the first side and the second side; a plurality of support posts; and a motor mount disposed adjacent to the opening, the motor mount including a protrusion extending from the first side of the support structure; and a motor retained within the motor mount, wherein the motor is retained in position by the motor mount and by the plurality of support posts.

In embodiments, the motor assembly further comprises a controller, wherein the controller is in electrical communication with the motor.

According to an aspect of the disclosure, a motor assembly may include: a support structure having a first side opposite a second side; a motor mount; a motor retained by the motor mount, the motor including a first electric terminal and a second electric terminal; and an interlocking mechanism attaching the motor mount to the support structure.

In embodiments, the motor assembly further comprises a controller in electrical communication with the motor. In embodiments, the interlocking mechanism includes: a first projection extending from the first side of the support structure, along the first side of motor mount, and over the motor mount; and a second projection extending from the first side of the support structure, along the second side of the motor mount, and over the motor mount to connect to the first projection. In embodiments, each of the first projection and the second projection is an elastically deforming member. In embodiments, the motor assembly further comprises a plurality of the first and second projections.

According to an aspect of the disclosure, a motor assembly may include: a support structure having a first side opposite a second side; a motor including a first electric terminal and a second electric terminal; a motor mount retaining the motor and including a first clamp and a second clamp; and a controller having a first portion opposite a second portion, wherein the controller is secured to the motor by the first clamp at the first portion of the controller, and wherein the motor and the controller are in electrical communication.

In embodiments, the first clamp and the second clamp each include a pair of a flexible engagement structures, wherein the first clamp at least partially encloses the first portion of the controller, and wherein the second clamp at least partially encloses the second portion of the controller.

According to an aspect of the disclosure, a motor assembly may include: a support structure having a first side opposite a second side; an opening defined in the support structure; a motor mount extending from the first side of the support structure; a motor retained by the motor mount, the motor including a first electric terminal and a second electric terminal; and a first connector and a second connector, wherein the first connector and the second connector extend into the opening defined in the support structure and are configured to form an electrical connection between the motor and a controller, and wherein the first connector and the second connector are retained in position by at least one extension of the motor mount.

In embodiments, the support structure includes studs adjacent to the opening in the support structure, and wherein the studs extend through holes defined in the first connector and the second connector. In embodiments, the motor mount includes at least two extensions, wherein the at least two extensions facilitate positional retention of the first connector and the second connector, or wherein the motor mount further includes inclined portions that facilitate positional retention of the first connector and the second connector. In embodiments, the first connector and the second connector are spaced by an insulating material. In embodiments, the motor mount includes a plurality of crush ribs positioned along inner walls of the motor mount. In embodiments, the motor mount includes a plurality of studs, wherein each stud of the plurality of studs is configured to extend through one of the first connector or the second connector and form an interference fit with the one of the first connector or the second connector.

### BRIEF DESCRIPTION OF THE DRAWINGS

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

Moreover, there are several embodiments described and illustrated herein. The present disclosure is neither limited to any single aspect or embodiment thereof, nor is the present disclosure to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein.

FIG. 1A shows a perspective view of a motor assembly.

FIG. 1B shows an enlarged, partial cross-sectional view of the motor assembly of FIG. 1A along line 1B.

FIG. 1C shows a perspective view of another example of a motor assembly.

FIG. 1D shows a perspective view of yet another example of a motor assembly.

FIG. 2 shows a cross-sectional view of a motor including a first electric terminal and a second electric terminal.

FIG. 3 shows a cross-sectional view of a motor assembly that utilizes and/or includes a spray conductor dispenser.

FIG. 4A and FIG. 4B each show a top view of a motor assembly.

FIG. 5A and FIG. 5B each show a top view of a motor assembly.

FIG. 6 shows a top view of a motor assembly.

FIG. 7 shows a perspective view of a motor assembly that includes a motor mount including a plurality of projections.

FIG. 8 shows a cross-sectional view of a motor assembly.

FIG. 9 shows a partial bottom view of a housing of a motor assembly.

FIGS. 10A-10D show a perspective view of a motor assembly.

FIGS. 11A-11D show a perspective view of a motor assembly.

FIG. 12 shows an enlarged, partial perspective view of a latch of a connector.

FIG. 13 shows an enlarged, partial perspective view of a recess.

FIG. 14 shows a side view of a connector including a latch.

FIG. 15 shows an enlarged, partial perspective view of a connector including a latch.

FIG. 16 shows a side view of a connector including a recess.

FIG. 17 shows an enlarged, partial cross-sectional view of a motor assembly.

FIG. 18 shows a side view of a connector.

FIG. 19 shows an enlarged, partial perspective view of a connecter including a recess.

FIG. 20A shows an enlarged, partial cross-sectional view of a motor assembly.

FIG. 20B shows an enlarged, partial side view of connector including a latch.

FIG. 21A shows a perspective view of a motor assembly including a plate.

FIG. 21B shows a partial side view of the plate of FIG. 21A.

FIG. 21C shows a perspective view of a motor assembly including a plate.

FIG. 22A and FIG. 22B show side, cross-sectional views of a motor assembly including a material between portions thereof.

FIG. 23 shows a support structure including an engagement structure.

FIG. 24 shows a side partial view of a flexible member.

FIGS. 25A and 25B show perspective views of a motor assembly including a connector.

FIG. 25C shows a side, cross-sectional view of a motor assembly.

FIG. 26 shows an enlarged, partial cross-sectional view of a motor assembly.

FIG. 27 shows a controller.

FIG. 28A shows a partial perspective view of a motor assembly.

FIG. 28B shows a partial rear view of the motor assembly of FIG. 28A.

FIG. 28C shows another partial perspective view of the motor assembly of FIG. 28A.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art would appreciate that the side views are not drawn to scale and should not be viewed as representing proportional relationships between different components. The side views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. In the discussion that follows, relative terms such as “about,” “substantially,” etc. may be used to indicate a possible variation of ±10% in a stated numeric value.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

Aspects of the disclosure relate to, among other things, motor assemblies and drug delivery devices associated therewith. Each of the aspects disclosed herein may include one or more of the features described in connection with any of the other disclosed aspects. It shall be understood that both the foregoing general description and the following detailed description are exemplary and explanatory only and are not restrictive of any claimed inventions.

The description herein and examples are illustrative and are not intended to be restrictive. One of ordinary skill in the art may make numerous modifications and/or changes without departing from the general scope of the invention. For example, aspects of above-described embodiments may be used in any suitable combination with each other. Additionally, portions of the embodiments described herein may be removed without departing from the scope of the invention. In addition, modifications may be made to adapt a particular situation or aspect to the teachings of the various embodiments without departing from their scope. Many other embodiments will also be apparent to those of skill in the art.

Various drug delivery devices, e.g., auto-injectors, utilize motors and/or motorized components during injection processes. It is advantageous for the components of such drug delivery devices to be structurally stable during operation of the overall drug delivery device. Such structural stability may aid in minimizing displacement of the individual components of the motor assembly during operation of the motor assembly, thereby preventing undesired mechanical or electrical decoupling of the individual components. The example motor assemblies described herein may be used during the manufacture and/or preparation of drug delivery devices, and the motor assemblies described herein facilitate the mechanical and electrical structural stability of such devices. Additionally, it is advantageous for the motors and/or motorized components of drug delivery devices to be configured such that the motors and/or motorized components are capable of being assembled/manufactured in an expedited manner (e.g., using a minimized number of steps). The example motor assemblies described herein may be assembled and/or manufactured using processes in which at least two features of a motor assembly are formed simultaneously, which facilitates the optimization of assembling/manufacturing the motor assembly.

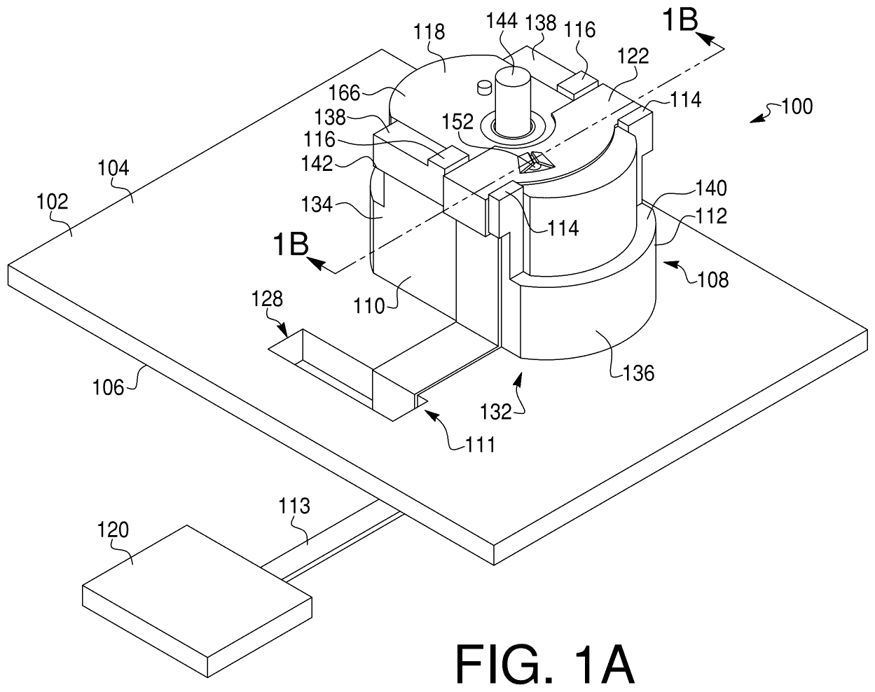

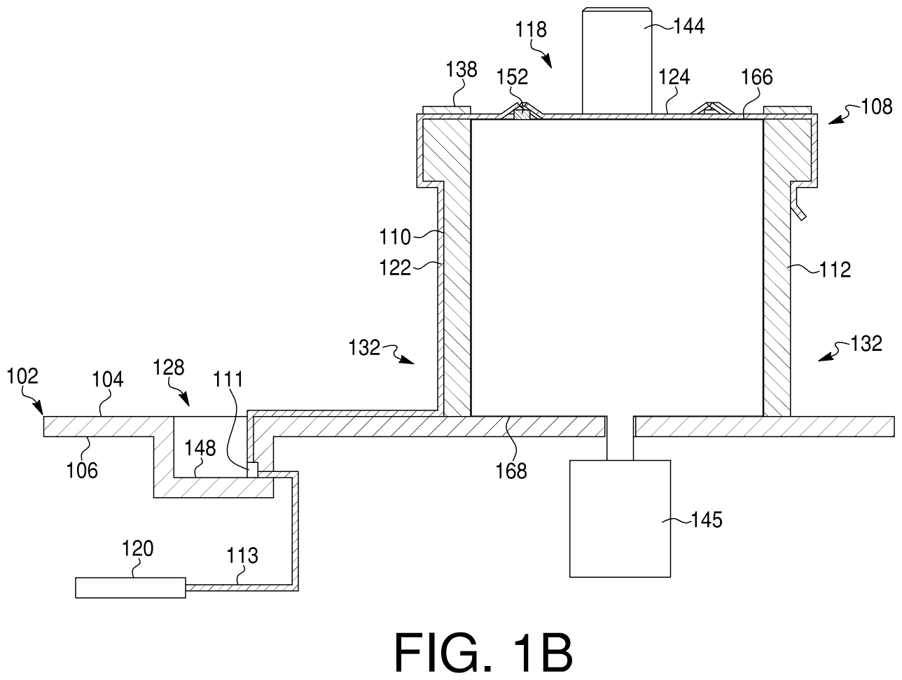

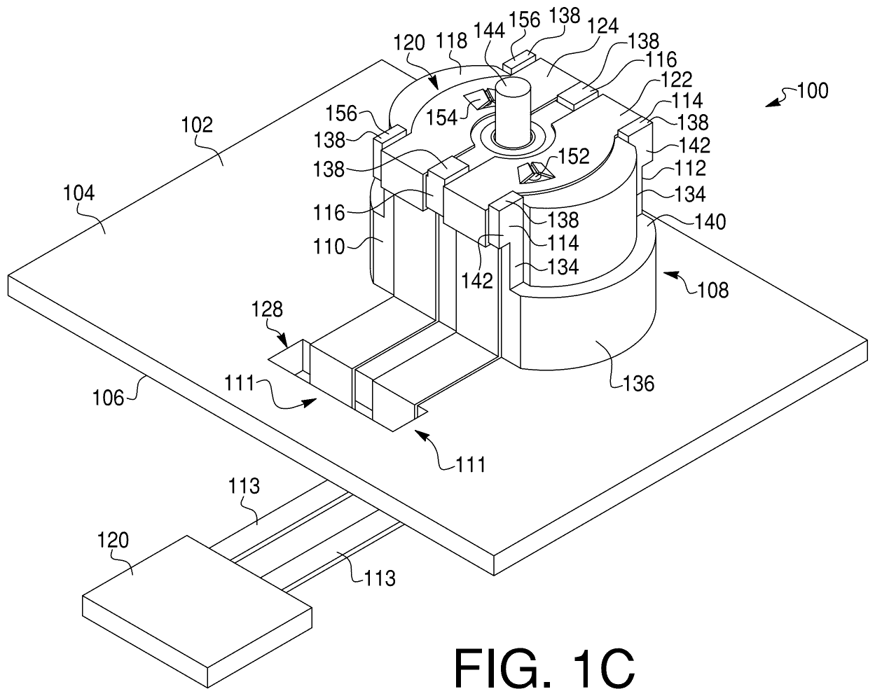

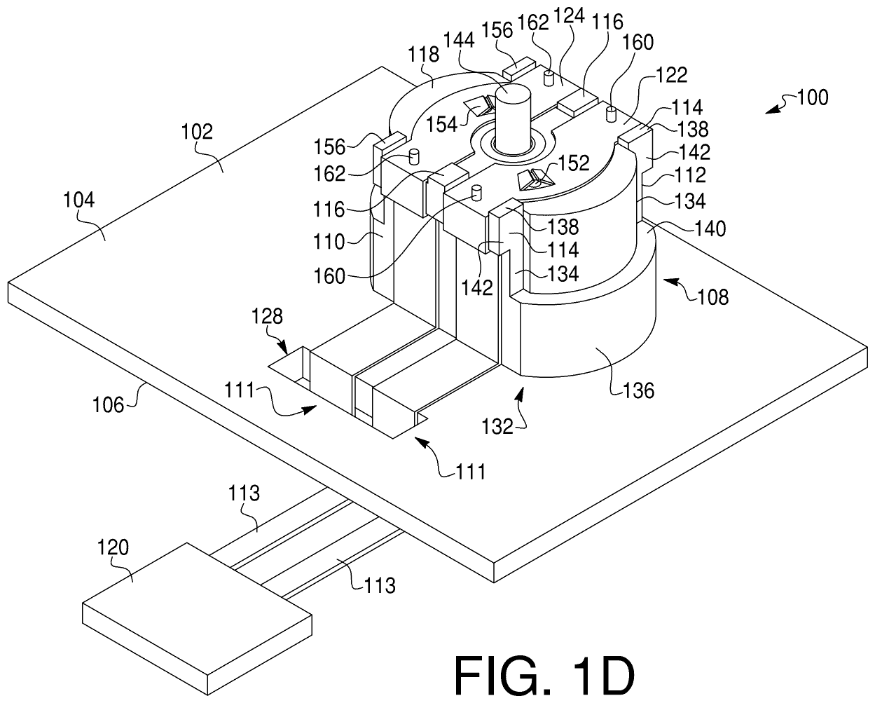

FIG. 1A shows a perspective view of a motor assembly 100, and FIG. 1B shows a (side) cross-sectional view of motor assembly 100. Motor assembly 100 may be included as part of a medicament delivery device, e.g., an auto-injector. Additional details of auto-injectors compatible with embodiments of the present disclosure may be found in U.S. Pat. No. 11,547,801 B2, entitled “AUTO-INJECTOR,” issued on Jan. 10, 2023, which is incorporated herein by reference in its entirety. Further details of auto-injectors compatible with embodiments of the present disclosure may be found in U.S. Patent Application Publication No. 2023-0149632 A1, published on May 18, 2023, which is incorporated herein by reference in its entirety.

Motor assembly 100 may include a support structure 102 having a first side 104 opposite a second side 106. Support structure 102 may be any structure appropriate for supporting the various components of motor assembly 100. For example, support structure 102 may be part of a housing of an auto-injector. As another example, support structure 102 may be an interior shelf of the housing of an auto-injector.

Suitable materials for support structure 102 include plastics, e.g., polycarbonate, acrylonitrile butadiene styrene, polypropylene, polyethylene; metals, e.g., stainless steel or aluminum; composites, e.g., carbon fiber reinforced polymer or glass reinforced polymer; biocompatible materials; medical grade silicone; or medical grade plastics, e.g., polyether ether ketone (also known as PEEK). It is understood that these example materials are non-limiting.

Motor assembly 100 may further include a motor mount 108. Motor mount 108 may be integrally formed with support structure 102, or motor mount 108 may be a separate component of motor assembly 100 that is attached to support structure 102. Motor mount 108 may include a protrusion 132 that extends from first side 104 of support structure 102. Protrusion 132 may include a first side 110 opposite a second side 112. Protrusion 132 may further include one or more linear portions 134 and one or more curved portion 136. In some embodiments, protrusion 132 may include all linear portions 134, giving motor mount 108 a polygonal shape, e.g., square, triangular, etc. In other embodiments, protrusion 132 may include all curved portions 136, giving motor mount 108 an oblong or circular shape. Linear portions 134 and/or curved portions 136 may define outer edges of motor mount 108. Motor mount 108 may include a surface 138 that defines a first raised portion 114 and a second raised portion 116 of motor mount 108, where first raised portion 114 and second raised portion 116 are disposed on either side of a connector 122. First raised portion 114 and second raised portion 116 may be adjacent to either or both of first side 110 and/or second side 112 of protrusion 132 of motor mount 108. First raised portion 114 and second raised portion 116 are considered to be “raised” because they extend above a planar surface 166 defined by a portion of the top of motor 118. Motor mount 108 may retain and/or enclose one or more portions of a motor 118, such as a shaft 144 of motor 118. Curved portion(s) 136 of protrusion 132 may include an upper surface 140. In some embodiments, protrusion 132 may include one or more extensions 142. Extensions 142 may extend laterally from linear portions 134 of protrusion 132 and may aid in retaining portions of connector 122 in place, e.g., during operation of motor assembly 100, by facilitating a tight fit of connector 122 and/or by minimizing movement of connector 122 during movement of shaft 144. The materials used for and/or included in motor mount 108 may be any of those described herein in reference to support structure 102.

As described, motor mount 108 may retain motor 118. Motor 118 may include various components, e.g., a stator, rotor, a shaft 144, and/or various other components of a motor that would be recognized by the skilled artisan. Motor 118 may generate rotational motion from stored energy and transmit the motion via shaft 144. Shaft 144 may rotate around a fixed axis, thereby moving, e.g., gears coupled or adjacent thereto. As shown in the side, cross-sectional view of FIG. 1B, shaft 144 of motor 118 may extend through a first surface 166 of motor 118, and/or shaft 144 may extend through second surface 168 of motor 118 opposite first surface 166, where the shaft 144 is in communication with and/or engaged to a spur gear 145. In an embodiment, shaft 144 may extend through first surface 166 of motor 118 and through second surface 168 of motor 118. In one embodiment, when motor assembly 100 is disposed within the housing of an auto-injector, shaft 144 may cause a needle to deploy and/or retract a needle used for injecting a medicament from a vial in the auto-injector into a patient.

Motor assembly 100 may include a controller 120. Controller 120, and each of the various controllers described herein, may be an electric control module or a printed circuit board (PCB). In some embodiments, controller 120 may be retained by support structure 102, while in other embodiments, controller 120 may be retained by another component (e.g., a separate housing). Controller 120 may be attached or retained by motor mount 108.

Controller 120 may be configured to control operation of motor 118, e.g., in terms of speed, direction, and/or torque of the motor 118. In some embodiments, controller 120 may control other functions of motor assembly 100, or controller 120 may control functions of an auto-injector including motor assembly 100. As examples, controller 120 may control injection speed, dose amount, dose depth, and dose temperature. In some embodiments, controller 120 may include power supply modules, microcontroller or microprocessor modules, sensor modules including temperature sensors, accelerometers, gyroscopes, humidity sensors, and proximity sensors, memory modules including random access memory (RAM) and flash memory, interface modules including USB interfaces, serial communication modules, and Ethernet modules, analog-to-digital and/or digital-to-analog converters, audio amplifiers, microphones, speakers, radiofrequency transmitters and receivers, and relays. In some embodiments, controller 120 may include wireless communications modules, including nearfield communications, Bluetooth, cellular modems, Wi-Fi modems, and/or global positioning systems (GPS) modules. Motor assembly 100 may further include a power source within, attached to, or external to the support structure 102, where the power source is electrically connected to controller 120 and/or to motor 118 of motor assembly 100. Controller 120 may include electric terminals, as will be described herein (terminals of controller 120 not shown in FIGS. 1A-1D).

As shown in FIG. 1A and FIG. 1B, controller 120 may be electrically coupled, either directly or indirectly, to motor 118 via a first connector 122 (and via a second connector 124, as will be discussed in reference to FIG. 1C and FIG. 1D). Controller 120 may be connected to electrical contact 111 (e.g., within opening 128 in support structure 102) via connection 113. Electrical contact 111 may further be connected to a terminal 152 of motor 118 via first connector 122, which at least partially places controller 120 into electrical communication with motor 118. It shall be understood that, while controller 120 is shown beneath second side 106 of support structure 102, controller 120 may alternatively be positioned above and/or coupled to first side 104 of support structure 102. As will be described in reference to FIGS. 1C and 1D, another electrical contact 111 may be connected to a second terminal 154 of motor 118 via second connector 124. Connection 113 may be formed, for example, with contact pads, pleated pads, wires, a flex cable, a ribbon cable, etc. Electrical contact 111 may be any suitable contact, such as a pin contact, blade contact, spring contact, tab contact, fork contact, box contact, receptacle contact, crimp contact, ribbon contact, or socket contact. Electrical contact 111 may include any suitable conductive material, including, but not limited to, copper, brass, phosphor bronze, beryllium, nickel silver, tin plated copper, gold plated metals, silver plated metals, aluminum, stainless steel, nickel, titanium, platinum, palladium, silicides, molybdenum, and tungsten. It is understood that electrical contact 111 may be disposed within opening 128 or external to opening 128, such that electrical contact 111 may have any suitable placement within or on motor assembly 100.

As mentioned, first connector 122 may mechanically secure motor 118 to motor mount 108. First connector 122 may include any suitable electrically conductive materials described herein, including metals. In embodiments, first connector 122 may be a structure that is mounted on surface 166 of motor mount 108. Portions of first connector 122 may be held in place by first raised portion 114 and second raised portion 116 of motor mount 108, as shown in FIG. 1A. First connector 122 may extend from second side 112 of motor mount 108, over motor mount 108, to first side 110 of motor mount 108, over first side 104 of support structure 102, and into opening 128 defined in support structure 102 to couple to electrical contact 111 therein. Opening 128 may be any suitable opening, e.g., a hole, a slot, a gap, etc., and may have any suitable shape, e.g., circular, square, rectangular, etc. Opening 128 may at least partially define a lower surface 148 of support structure 102, as shown in FIG. 1B. First connector 122 may be coupled to electrical contact 111, e.g., using a latch and a recess, as will be discussed in more detail herein.

As mentioned, motor 118 may include a first electric terminal 152 (and a second electric terminal 154, as shown in FIGS. 1C and 1D). First and second electric terminals 152, 154 of motor 118 may respectively extend through portions of first connector 122 (and through portions of second connector 124, as shown in FIGS. 1C and 1D) and through surface 166 of motor 118. First and second terminals 152, 154 may be any appropriate type of terminal, including, but not limited to, pin contacts, blade contacts, spring contacts, tabs contacts, fork contacts, box contacts, receptacle contacts, crimp contacts, ribbon contacts, or socket contacts. First and second terminals 152, 154 may include any appropriate conductive material discussed in reference to electrical contact 111. First and second terminals 152 and 154 may have opposite polarities (i.e., one positive terminal 152 or 154 and one negative terminal 154 or 152). In some embodiments, terminals 152 and 154 may be stamped on a side of motor 118 (or stamped onto a side of motor mount 108 and/or controller 120). In some embodiments, an isolation material (e.g., a dielectric such as a silicon oxide) may be between terminals 152 and 154. Terminals 152, 154 may be formed using any appropriate additive or subtractive manufacturing process. Suitable example additive processes for forming terminals 152, 154 may include sputtering, metal vapor deposition, and plating techniques. Suitable subtractive processes for forming terminals 152, 154 may include etching, machining, and laser-based techniques.

Referring now to both FIGS. 1C-1D, in some embodiments, motor assembly 100 includes a first connector 122 and a second connector 124. First connector 122 and second connector 124 may respectively extend from first side 110 of motor mount 108, over motor mount 108, to second side 112 of motor mount 108, over second side 106 of support structure 102, and into opening 128 defined in support structure 102 to couple a respective electrical contact 111 (and thus first connector 122 and second connector 124 may indirectly couple motor 118 to controller 120). Each electrical contact 111 may be coupled to controller 120 via a separate connection 113. Motor mount 108 may include a third raised portion 156, where second raised portion 116 and third raised portion 156 of motor mount 108 are disposed on either side of second connector 124. Raised portion 156 may aid in retaining second connector 124 in position, e.g., during operation of motor 118, by providing a tight fit for second connector 124 and/or by minimizing displacement of second connector 124 during rotation of shaft 144 of motor 118. First and second electric terminals 152, 154 may respectively be included in (or extend through) first connector 122 and second connector 124.

As shown in FIG. 1D, motor mount 108 may further include a first post 160 between first raised portion 114 and second raised portion 116 of motor mount 108, and a second post 162 between second raised portion 116 and third raised portion 156 of motor mount 108. Post 160 and 162 may extend from surfaces 138 of motor mount 108. Posts 160 and 162 may be substantially similar to one other. Posts 160, 162 may have any appropriate geometry, e.g., polygonal, cylindrical, etc. Posts 160, 162 may include any appropriate material, such as those described herein in reference to motor mount 108, first and second connectors 122, 124, and support structure 102. In some embodiments, posts 160, 162 may have the same material composition as motor mount 108. In other embodiments, posts 160, 162 may have a different material composition than motor mount 108. As shown in FIG. 1D, first post 160 may aid in retaining first connector 122 in place by extending through a hole defined in first connector 122. Second post 162 may aid in positional retention of second connector 124 by extending through a hole defined in second connector 124. That is, posts 160, 162 may align with holes in first connector 122 and in second connector 124 and may help align or retain first connector 124 and second connector 124 between first raised portion 114 and second raised portion 116 (or between second raised portion 116 and third raised portion 156).

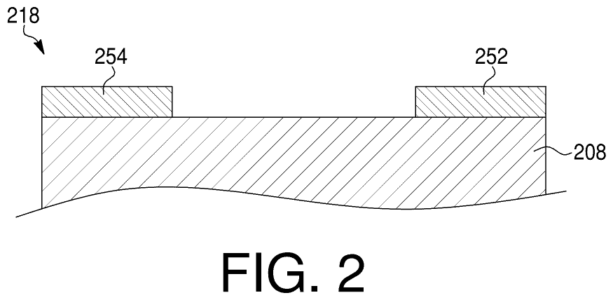

FIG. 2 shows an enlarged, partial cross-sectional view of a portion of a motor 218. Motor 218 may include a first electric terminal 252 and a second electric terminal 254, among other components. First and second electric terminals 252, 254 of motor 218 may be any suitable example of a terminal described herein. First and second electric terminals 252, 254 of motor 218 may be attached to (or may extend through) a surface of a motor mount of a motor assembly. Such a motor assembly may include a controller connected to motor 218. Terminals 252, 254 may include any appropriate conductive material described herein. In some embodiments, the motor assembly may include a nonconductive structure formed of plastic or another material that is used as a spacer between terminal 252 and 254 and/or between terminal 252, 254 and another component. In some embodiments, motor assembly 200 and/or terminals 252, 254 may include additively-formed conductors. In some embodiments, a spray conductor may be deposited on terminals 252 and 254. In some embodiments, terminals 252 and 254 may be formed via sputtering. In some embodiments, terminals 252 and 254 may be formed via metal vapor deposition or plating. In some embodiments, terminals 252 and 254 may be formed by subtractive manufacturing, e.g., formed as a single or continuous layer, and then material may be removed via, e.g., photolithography or etching. In some embodiments, terminals 252 and 254 may be formed through machining or laser sintering.

Terminals 252, 254 may have any appropriate cross-sectional shape, e.g., square, circular, triangular, etc. Terminals 252, 254 may be any appropriate type of terminal, including, but not limited to, pin contacts, blade contacts, spring contacts, tabs contacts, fork contacts, box contacts, receptacle contacts, crimp contacts, ribbon contacts, or socket contacts.

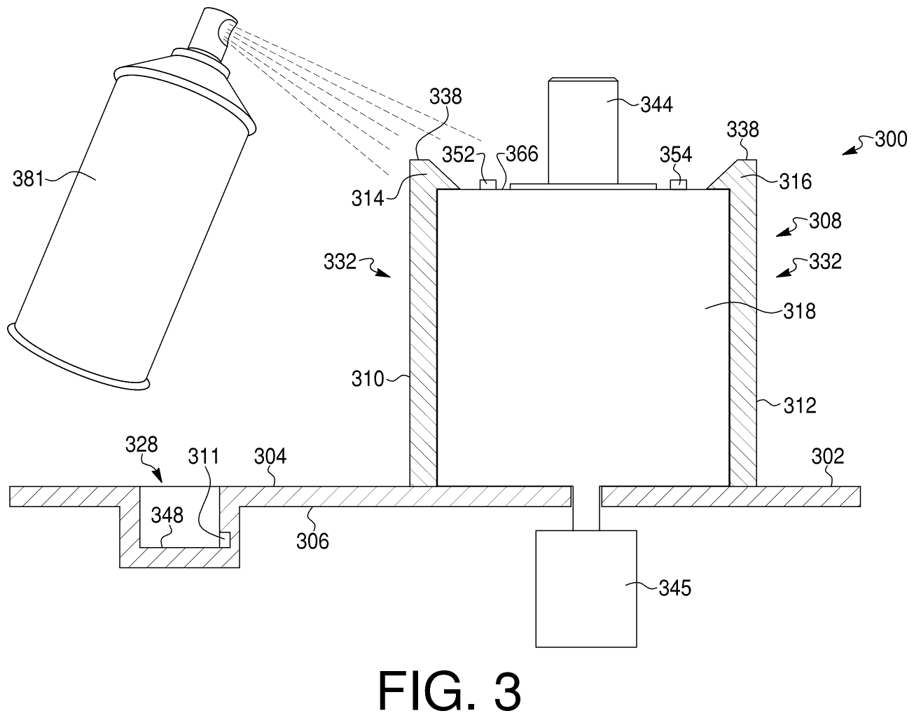

FIG. 3 shows a side view of a motor assembly 300. Motor assembly 300 may be substantially similar to other motor assemblies described herein. Motor assembly 300 may include a support structure 302 including a first side 304 opposite a second side 306 and may further include a motor mount 308. Motor mount 308 may include a first side 310 and a second side 312 and may retain a motor 318. Motor mount 308 may include a protrusion 332. Protrusion 332 may have linear portions and/or curved portions, similar to protrusion 132 described in reference to FIGS. 1A-1D. Portions of a surface 338 of motor mount 308 may defined raised portions 314, 316 of motor mount 308, where raised portions 314, 316 extend above a planar surface 366 defined by at least another portion of (a top of) motor mount 308. Motor assembly may include a controller (e.g., a printed circuit board) that is in electrical communication with motor 318. The controller (not shown in FIG. 3) may be coupled to an electrical contact 311 within an opening 328 in support structure 302 (or anywhere on support structure 302), and electrical contact 311 may be coupled to terminals 352, 354 of motor 318 using a conductive material from a spray conductor dispenser 381. Masking materials may be used during application of the conductive material from spray conductor dispenser 381, e.g., to ensure that each terminal 352, 354 is independently coupled to a respective electrical contact 311. In some embodiments, there may be a lower surface 348 in support structure 302 that is at least partially defined by opening 328. Motor 318, which may be retained by motor mount 308, may include a shaft 344 in communication with and/or engaged to a spur gear 345. Motor assembly 300 further includes first and second electric terminals 352, 354, which may be included on a controller, on motor 318, or on motor mount 308. Spray conductor dispenser 381 may utilize and/or include metal particles, e.g., copper, brass, phosphor bronze, beryllium, nickel silver, tin plated copper, gold plated metals, silver plated metals, aluminum, stainless steel, nickel, titanium, platinum, palladium, silicides, molybdenum, and tungsten.

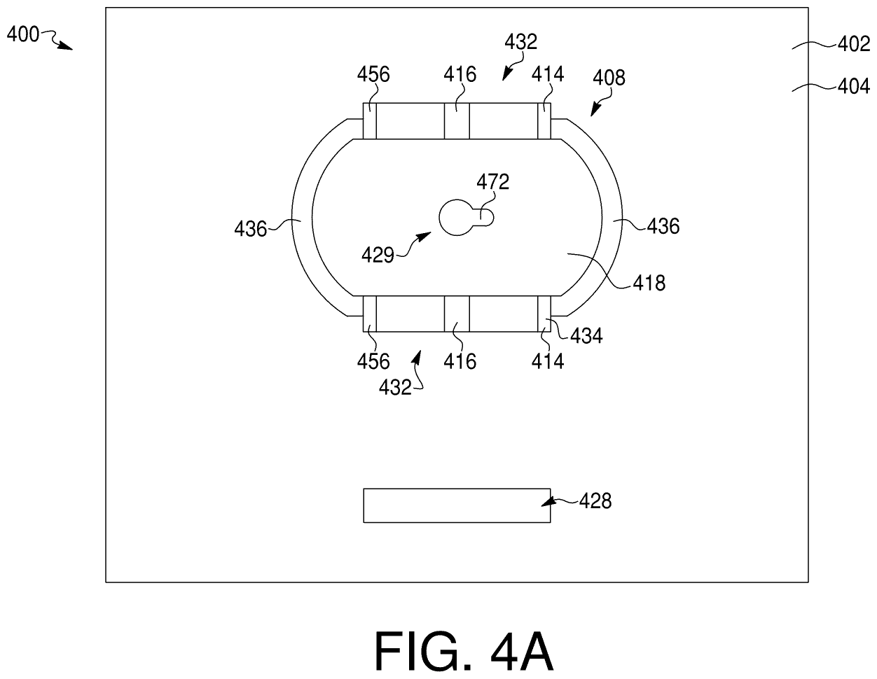

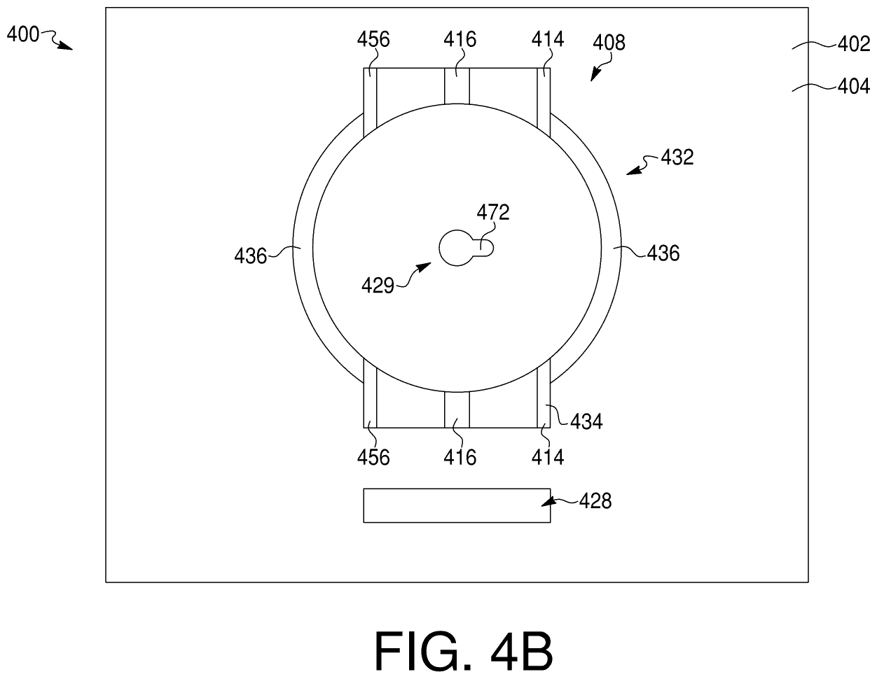

FIGS. 4A-4B show top views of different configurations of a motor assembly 400. Motor assembly 400 may include similar features or components as other motor assemblies described herein unless otherwise specified.

Motor assembly 400 may include a support structure 402 including a surface 404 and may further include a motor mount 408. In some embodiments, and as shown in FIG. 4A and FIG. 4B, support structure 402 may include a motor shaft opening 429 that is surrounded by motor mount 408 and another opening 428 is adjacent to motor mount 408. Motor mount 408 may include a protrusion 432 including raised surfaces 414, 416, and 456. Protrusion 432 may include linear portions 434 and curved portions 436, similar to protrusion 132 (described in reference to FIGS. 1A-1D). In some embodiments, a support post of any motor described herein (not shown in FIGS. 4A-4B) may extend through (or be positioned adjacent to) opening 429 in support structure 402. The post and opening 429 may be configured such that when the post is in a first position, motor 418 is removable from opening 429 (and thus motor 418 is removable from support structure 402). The post and opening 428 may be configured such that when the post is in a second position, the post may be held in place within opening 429 (and thus motor 418 is locked into place in support structure 402). In some embodiments, the post extends through opening 428 and, when the motor 418 is rotated, a protrusion on the post is no longer aligned with the hole, thereby securing motor 418 therein. In some embodiments, opening 429 may be any appropriate shape, e.g., circular, square, triangular. In other embodiments, opening 429 may have a non-polygonal shape, e.g., circular, elliptical. In some embodiments, opening 429 may have a circular portion with an extended edge 472 (e.g., similar to a keyhole shape). In some embodiments, a controller may be in electrical communication with a motor when the post of the motor is in the second position within opening 428.

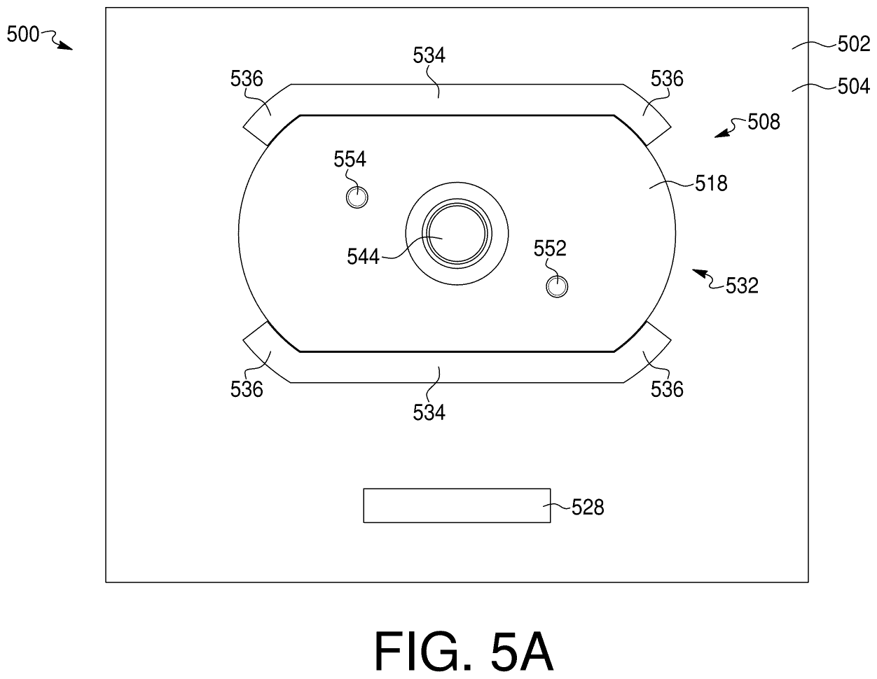

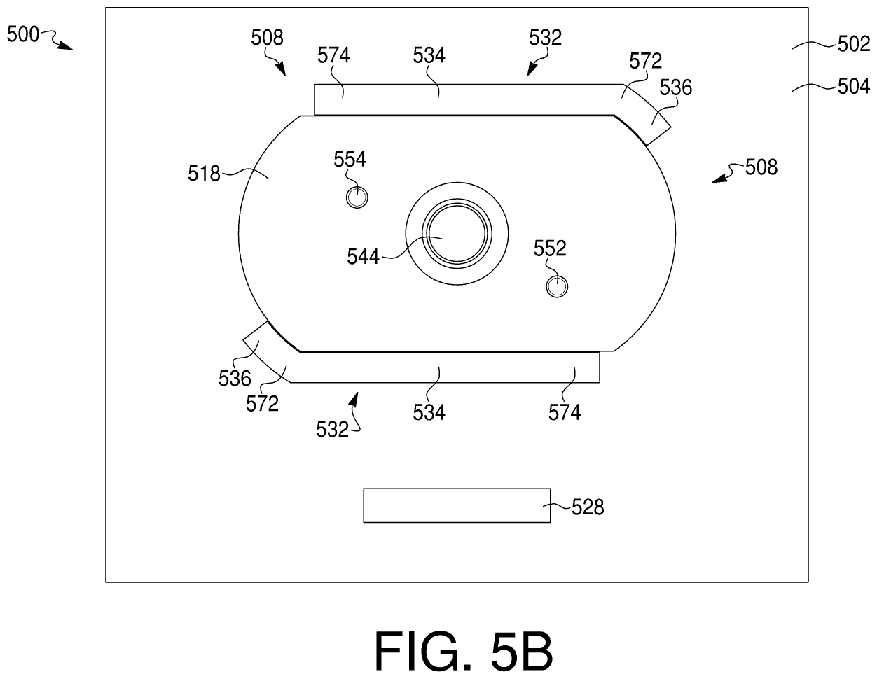

FIGS. 5A and 5B show top views of a motor assembly 500. Motor assembly 500 may include support structure 502 including a surface 504, and a motor mount 508 thereon. Motor mount 508 may include linear portions 534 and curved portions 536, as discussed in reference to the other protrusions described herein. Motor assembly 500 may include a motor 518, including a shaft 544 and terminals 552 and 554. Terminals 552, 554 may be any appropriate type of terminal, including the types described above. Terminals 552 and 554 may be electrically coupled to a motor, such as those described herein, to a controller, e.g., via an intervening electrical connection as described in reference to motor assembly 100.

Protrusion 532 of motor mount 508 may extend from a support structure 502. In some embodiments and as shown in FIG. 5B, protrusion 532 may have a first side 572 opposite a second side 574, and protrusion 532 may include curved portions 536 on each of first side 572 and second side 574. In some embodiments and as shown in FIG. 5B, protrusion 532 have only one curved portion 536 at either first side 572 or second side 574. Curved portions 536 of protrusions 532 may be on opposite sides from one another, as shown in FIG. 5B, e.g., curved portion 536 may be on first side 572 of one protrusion 532 while another curved portion 536 may be on second side 574 of protrusion 532.

Motor assembly 500 may further include an opening 528 in support structure 502, which may be substantially similar to the various openings in support structures described herein (e.g., opening 128 in FIGS. 1A-1D). In some embodiments, a support post of motor 518 may extend through a motor shaft opening in support structure 502 (e.g., similar to the opening 429 discussed in reference to FIG. 4A and FIG. 4B). The post and the opening may be configured such that when the post is in a first position, motor 518 is removable from the opening (and thus from support structure 502). The post and the opening may be configured such that when the post is in a second position, motor 518 is held in place within opening 528. In some embodiments, a controller (e.g., a printed circuit board) may be in electrical communication with motor 518 when the post of motor 518 is in the second position within the opening.

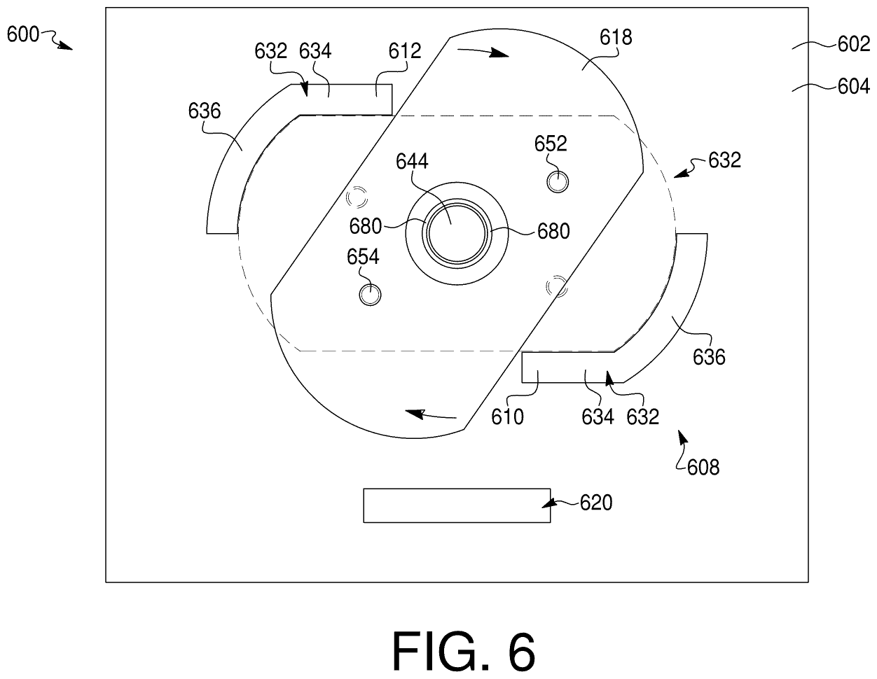

FIG. 6 shows a top view of a motor assembly 600. Motor assembly 600 may include a support structure 602 including a surface 604 and a motor mount 608 thereon. Motor mount 608 may include a first side 610 opposite a second side 612. Motor assembly 600 may include a motor 618 retained therein. Motor 618 may include a shaft 644 and terminals 652 and 654. Motor mount 608 may include a protrusion 632. Protrusion 632 may include linear portions 634 and/or curved portions 636. In some embodiments, a motor shaft opening defined through motor mount 608 and defined through support structure 602 (similar to motor shaft opening 429 discussed in reference to FIGS. 4A and 4B) may be threaded on respective interior surfaces 680, e.g., to accommodate the rotational movement of a shaft of motor 618. In some embodiments, motor 618 may couple with threading on surfaces 680 of protrusion 632 through threading on an exterior of motor 618. In some embodiments, motor 618 may be secured within protrusion 632 of motor mount 608 by rotating motor 618 within motor mount 608. Motor 618 and/or motor mount 608 may be in electrical communication with a controller 620.

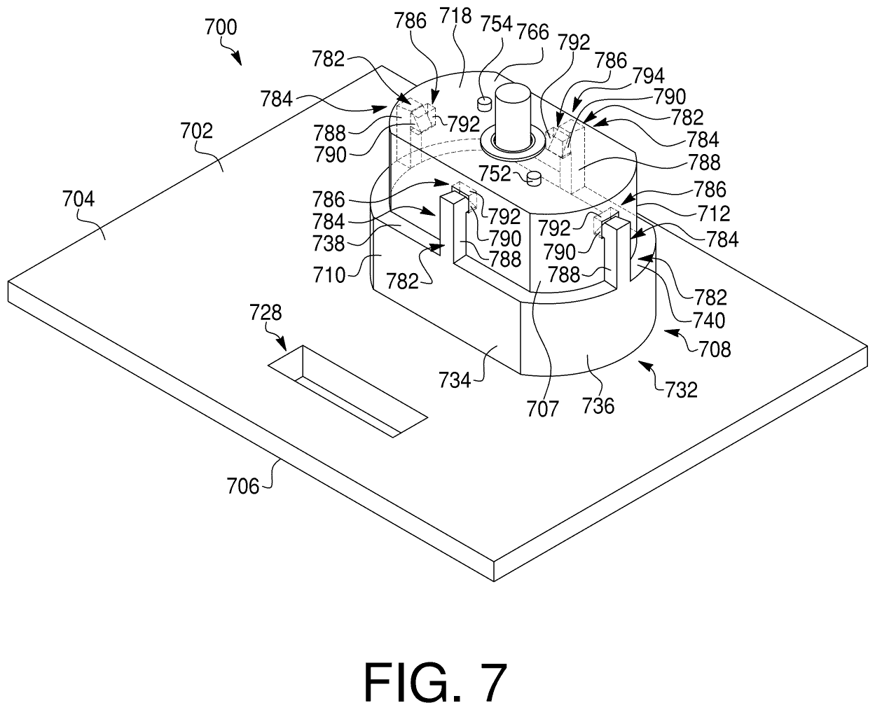

FIG. 7 shows a perspective view of a motor assembly 700. Motor assembly 700 may include a support structure 702 including a first side 704 opposite a second side 706. Motor assembly 700 may include an opening 728 in support structure 702. Motor assembly 700 may include a motor mount 708. Motor 718 may be retained by motor mount 708 and may include a first portion 784 and a second portion 786 of an interlocking mechanism 782. Second portion 786 of interlocking mechanism 782 may be configured to mechanically couple to first portion 784 of interlocking mechanism 782 to secure motor 718. In some embodiments, second portion 786 of interlocking mechanism 782 may include terminals (e.g., any of the suitable terminals and/or conductive materials described herein) and first portion 784 may include conductive material for forming an electrical connection between the terminals and a controller. Motor mount 708 may include a first side 710 opposite a second side 712 and a protrusion 732. Protrusion 732 of motor mount 708 may include linear portions 734 and/or curved portions 736. Linear portions 734 may include a surface 738 and curved portions may include a surface 740. Motor assembly 700 may include a motor 718 similar or the same as other motors described herein. Motor 718 may include terminals 752 and 754, e.g., that are attached to, or that extend through a surface 766 of, motor 718. Terminals 752, 754 may be any appropriate type of terminal described above.

In an embodiment shown in FIG. 7, first portion 784 of interlocking mechanism 782 may include a projection 788 extending from surface 740 of curved portions 736 of protrusion 732. In some embodiments, projection 788 may include an engagement structure 790 extending from projection 788, where engagement structure 790 mechanically couples to a recess 792 of second portion 786 of interlocking mechanism 782. Recess 792 may have any appropriate shape, e.g., circular, square, triangular, elliptical. In some embodiments, recess 792 may extend fully or partially through motor 718. Projection 788 may be a flexible arm configured to allow insertion and removal of engagement structure 790 within recess 792. In some embodiments, recess 792 has a non-polygonal shape. In some embodiments, motor 718 and controller 120 are in electrical communication when first portion 784 is mechanically engaged to second portion 786. That is, in some embodiments, first portion 784 may form an electrical connection with second portion 786 when portions 784 and 786 are engaged, e.g., mechanically coupled. While not explicitly shown in FIG. 7, in some embodiments, when interlocking mechanism 782 is engaged, motor 718 and a controller, such as a printed circuit board, are in communication.

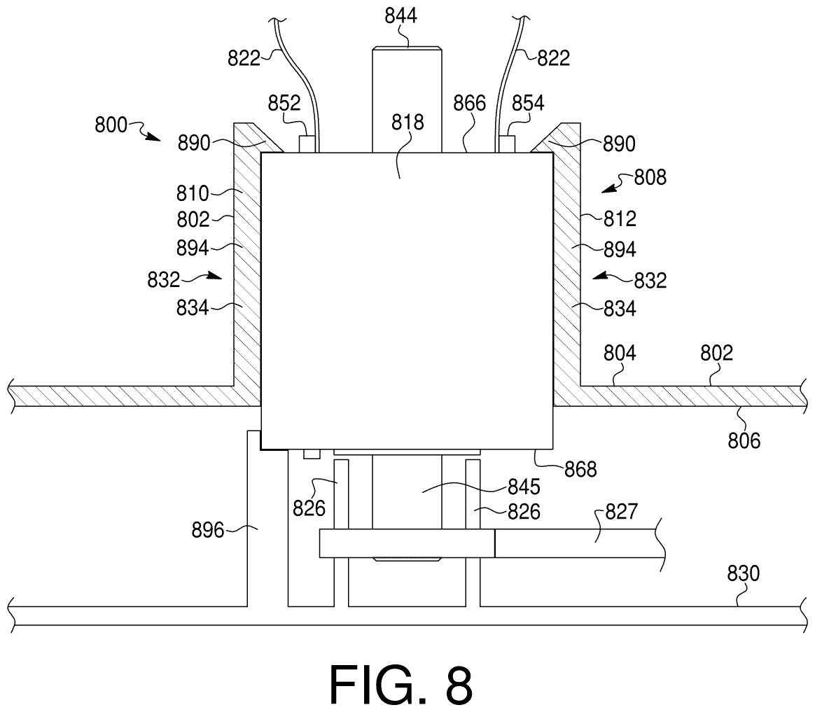

FIG. 8 shows a side cross-sectional view of a motor assembly 800. Motor assembly 800 may include a support structure 802 having a first side 804 opposite a second side 806. Motor assembly 800 may include a motor mount 808, which may include a first side 810 opposite a second side 812. Motor mount 808 may include a protrusion 832, which may include linear portions 834 (and/or curved portions). Motor 818 (retained by mount 808) may have a surface 866 that at least partially defines raised portions thereof. Surface 866 of motor 818 may include a first electric terminal 852 and a second electric terminal 854 (of motor 818) thereon or extending therethrough. Connectors 822 may extend from terminals 852, 854 of motor 818 to, e.g., a controller (not shown in FIG. 8). Engagement structures 890 may extend over a surface 866 of motor 818, thereby securing motor 818 within motor mount 808, and securing motor mount 808 to support structure 802. Support structure 802 may include a support post 896 to mechanically secure motor 818 to support structure 802. Support structure 802 may include support posts 826 near shaft 844 to aid in retaining the position of motor 818 during its operation. In some embodiments, a shaft 844 may extend from a second surface 868 of motor 818 to first surface 866 of motor 818. Shaft 844 may be in communication with and/or engaged to a spur gear 845. Spur gear 845 may engage with a transmission assembly 827, which may include one or more additional gears configured to cause movement of components of an injector. In some embodiments, motor assembly 800 may include a housing 830 on which motor 818 may be mounted, e.g., through support posts 826 and 896.

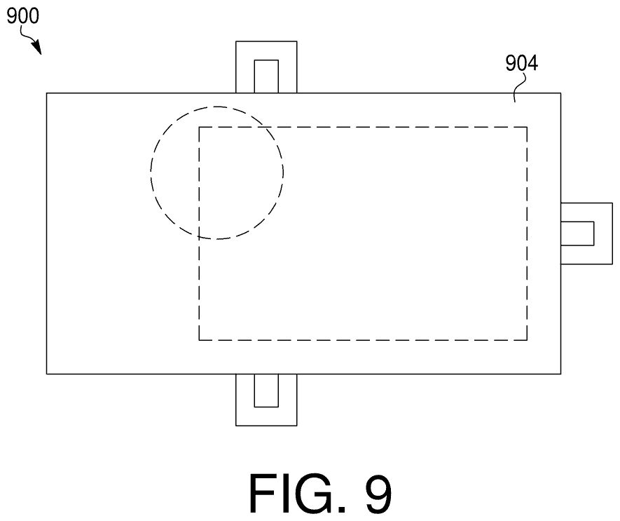

FIG. 9 shows a bottom partial view of a housing 900. Housing 900 may include a surface 904 that is opposite a surface with a motor assembly extending therefrom (surface with motor assembly not shown in FIG. 9). That is, a motor assembly may be on a second surface (not shown) opposite the surface 904 and on an interior of an auto-injector housing (represented by the dashed line circle in FIG. 9). In embodiments, surface 904 of housing 900 may face a patient or user of an auto-injector. In embodiments, the positioning of the motor assembly on the second surface of housing 900 facilitates the inclusion of the motor assembly in an auto-injector without the use of an enclosing cover. Thus, in embodiments, use of the housing 900 in combination with the motor assembly reduces the number of overall components that are utilized during the preparation of an auto-injector.

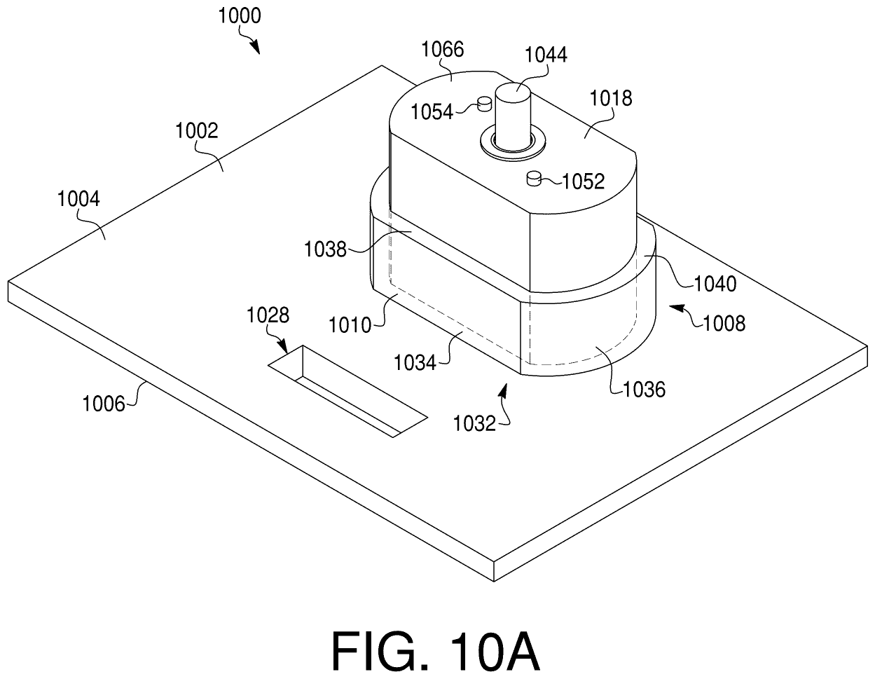

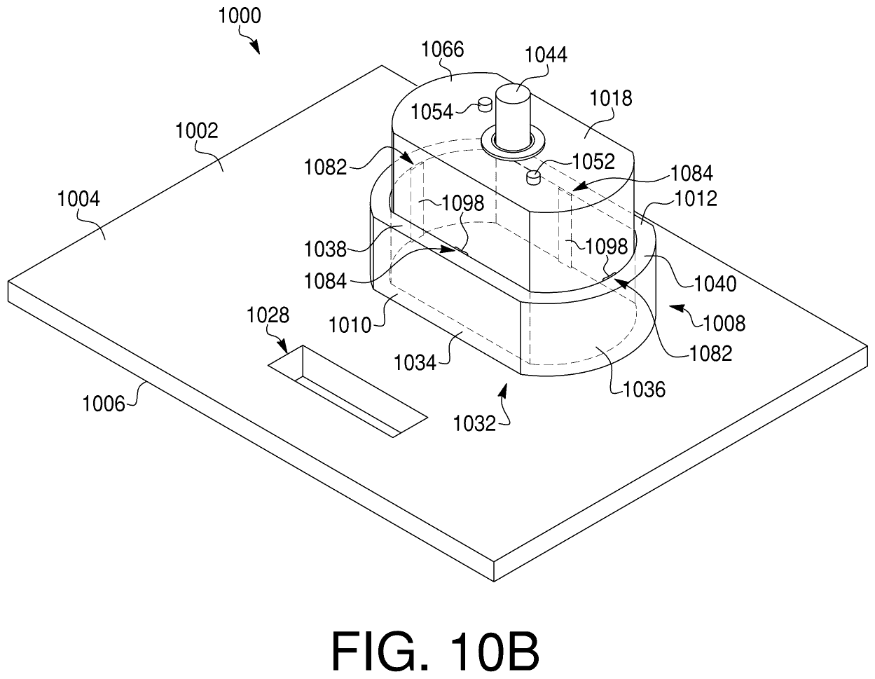

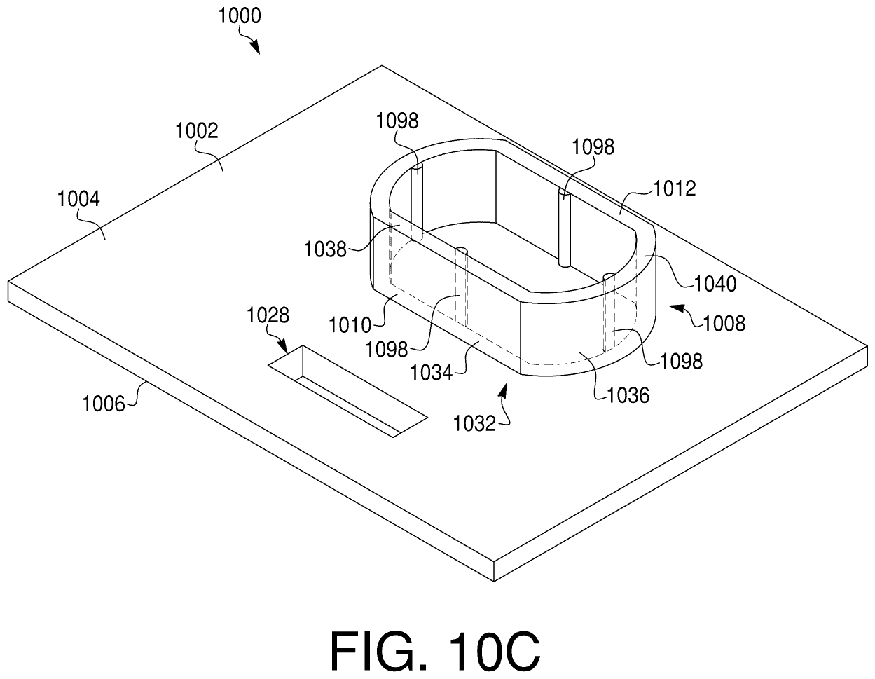

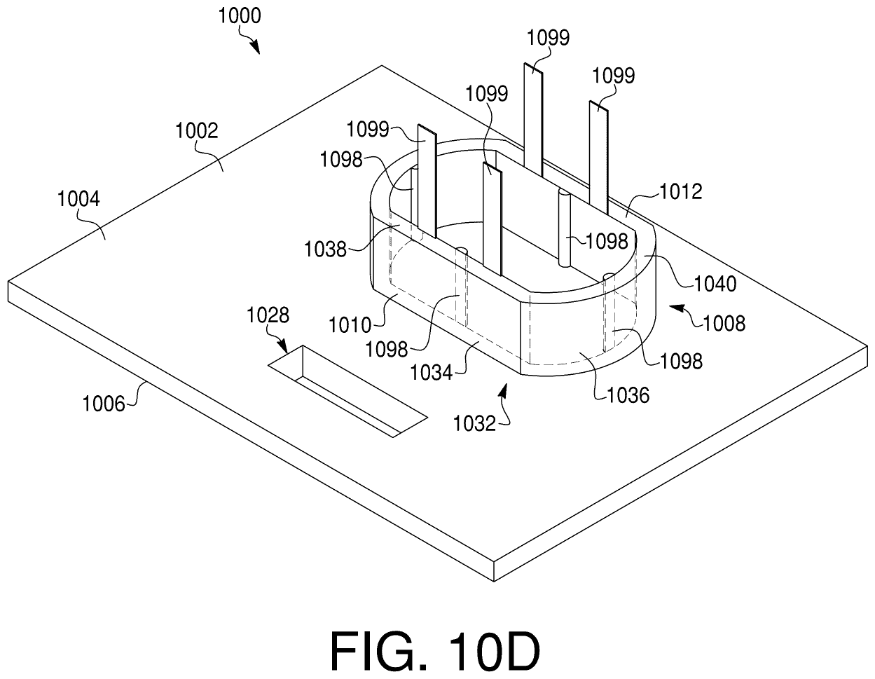

FIGS. 10A-10D show perspective views of a motor assembly 1000. Motor assembly 1000 may include a support structure 1002 including a first side 1004 opposite a second side 1006. Support structure 1002 may include an opening 1028. As shown in FIG. 10A, motor assembly 1000 may include a motor mount 1008. Motor mount 1008 may include a first side 1010 opposite a second side 1012, and a protrusion 1032. Protrusion 1032 may include linear portions 1034 and curved portions 1036. Linear portions may include surfaces 1038 and curved portions 1036 may include surfaces 1040. Motor assembly 1000 may include a motor 1018, including terminals 1052 and 1054 on a surface 1066 of motor 1018, and a shaft 1044 extending from surface 1066. Terminals 1052, 1054 may be any appropriate type of terminal, including, but not limited to, pin contacts, blade contacts, spring contacts, tabs contacts, fork contacts, box contacts, receptacle contacts, crimp contacts, ribbon contacts, or socket contacts. Terminals 1052, 1054 may include any appropriate conductive material described herein.

As shown in FIG. 10B, motor mount 1008 of motor assembly 1000 includes a plurality of wall members 1098. Wall members 1098 may be included on each inner wall of motor mount 1008. Wall members have properties of elasticity and may be configured to compress and exert force on walls of motor 1018 during operation of motor 1018, which may aid in retaining motor 1018 in place. In some embodiments, wall members 1098 may be crush ribs positioned along each inner wall of motor mount 1008 and may be configured to deform and/or compress upon contact with motor 1018.

FIG. 10C shows motor mount 1008 without motor 1018 retained thereby, e.g., to further illustrate the various components of motor assembly 1000.

FIG. 10D shows a motor assembly 1000 including deforming members 1099 in an unbent state. Deforming members 1099 may be bent inward (i.e., toward the middle of motor mount 1008) to aid in securing motor mount 1008 to support structure 1002. In some embodiments, motor mount 1008 may be configured to provide an interference fit to retain motor 1018.

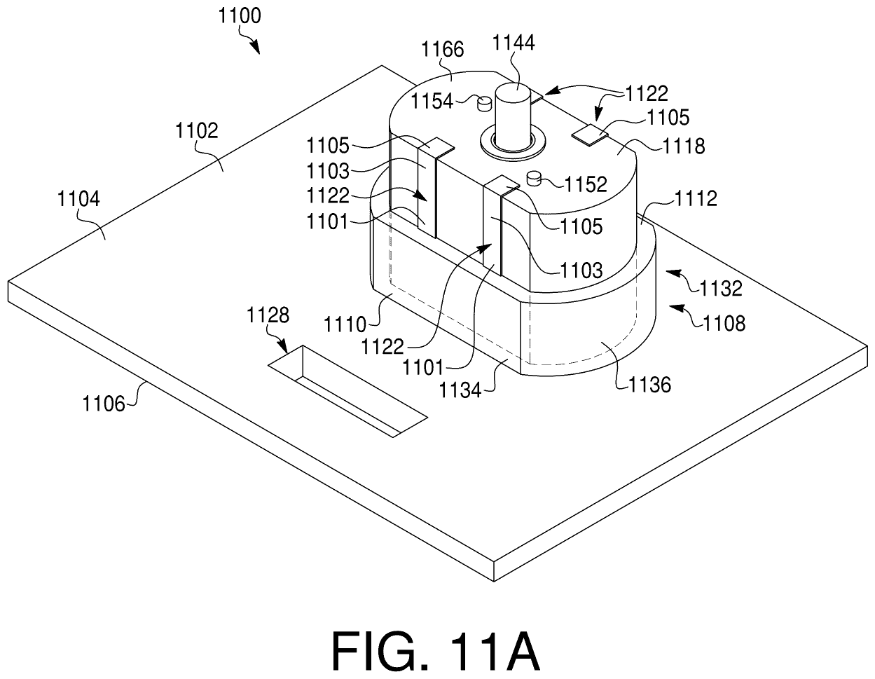

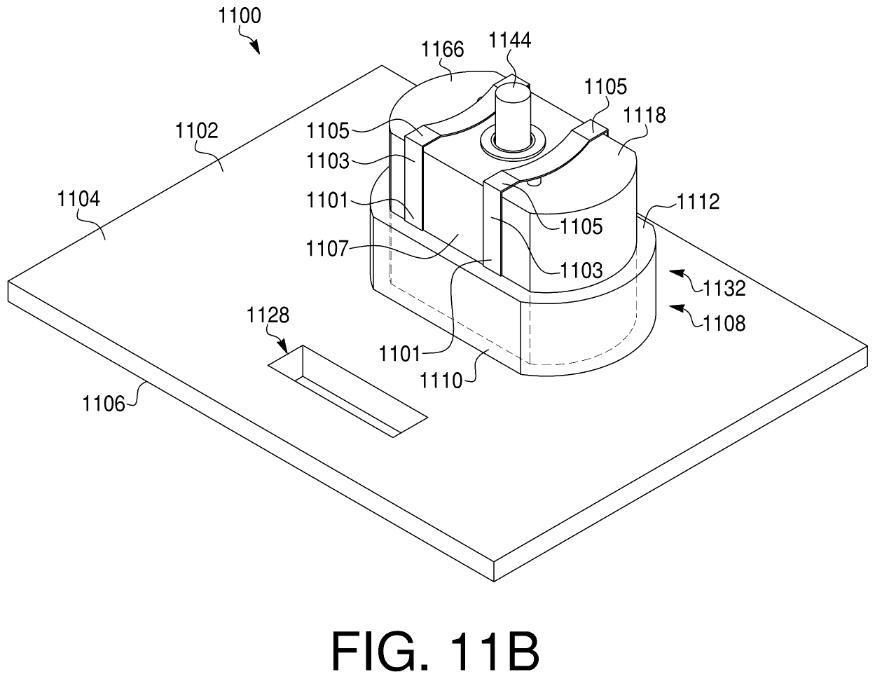

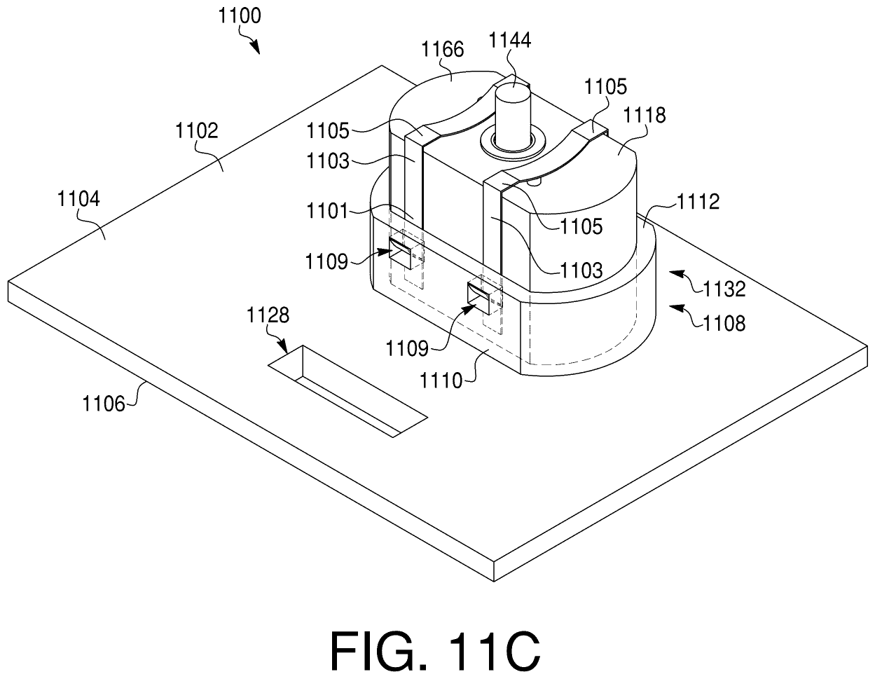

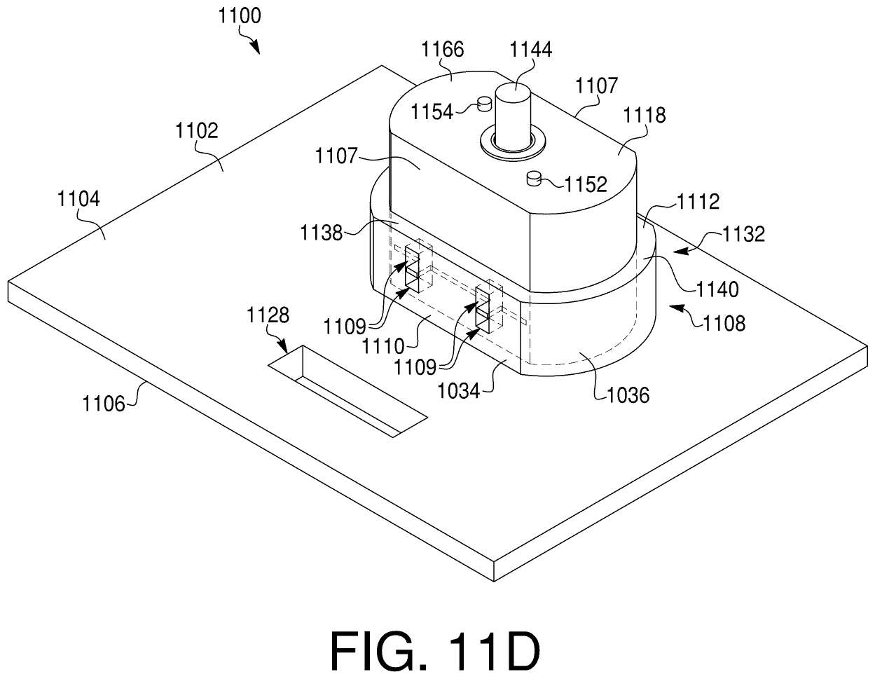

FIGS. 11A-11D show perspective views of a motor assembly 1100. Motor assembly 1100 may include a support structure 1102 including a first side 1104 opposite a second side 1106. In some embodiments, support structure 1102 may include an opening 1128. Motor assembly 1100 may further include a motor mount 1108 on support structure 1102. Motor mount 1108 may include a first side 1110 opposite a second side 1112. Motor mount may include linear portions 1134 and curved portions 1136. In some embodiments, linear portion 1134 may include a surface 1138, and curved portion 1136 may include a surface 1140. Motor assembly 1100 may include a motor 1118 retained by motor mount 1108, where motor 1118 includes first and second electric terminals 1152, 1154. In some embodiments, motor 1118 may include a shaft 1144.

Connector(s) 1122 may retain motor 1118 within motor mount 1108, and/or connector(s) 1122 may retain motor mount 1108 to first side 1104 of support structure 1102. Connector 1122 may be made of or include any appropriate material described herein. Connector 1122 may be at least partially formed using deforming members, as described in reference to FIGS. 10A-10D.

In some embodiments, motor assembly 1100 may include one, two, three, four, or more connectors 1122. Connectors 1122 may each have a first portion 1101 connected to an inner wall of motor mount 1108 and a second portion 1103 that extends along first side 1110 or second side 1112 of motor mount 1108. Connectors 1122 may include a third portion 1105 on surface 1166 of motor 1118. Connectors 1122 may be positioned over first and second electric terminals positioned on motor mount 1108. In an embodiment, and as shown in FIG. 11C, first portion 1101 of connector 1122 is coupled to a recess 1109 in motor mount 1108. Motor mount 1108 may include a plurality of recesses 1109. In some embodiments, recess 1109 may be any appropriate shape, e.g., circle, square, oblong. Recess 1109 may extend through protrusion 1132 of motor mount 1108. In different embodiments, connectors 1122 may be reversibly or irreversibly connected to recesses 1109, as shown in FIGS. 11C and 11D. As discussed in reference to the other motor assemblies described herein, connectors 1122 may be individually coupled to a terminal (of a motor 1118) and to an electrical connection, e.g., that is positioned in opening 1128 in support structure 1102. Further, the electrical connection may be coupled to a controller. Thus, connectors 1122 may place a controller in electrical communication with motor 1118.

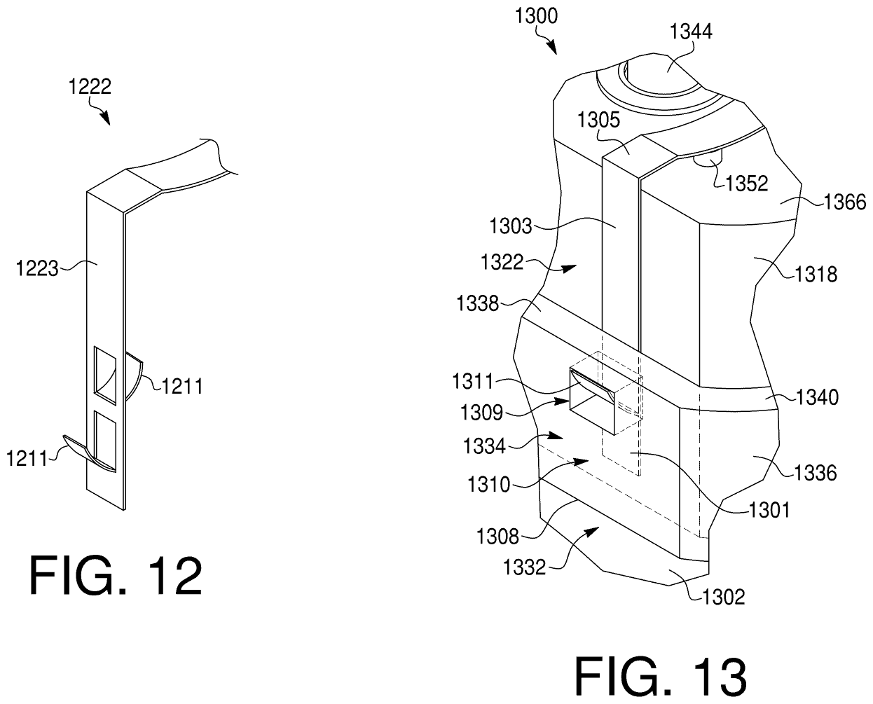

FIG. 12 shows an enlarged partial perspective view of a connector 1222. Connector 1222 may be similar to other connectors described herein and may include latches 1211 extending inwardly and/or outwardly from a body 1223 of connector 1222. In some embodiments, connector 1222 may include one, two, three, four, five, or more latches 1211. Latches 1211 may be used to couple connectors 1222 to recesses, such as those described in other embodiments herein, to secure or retain a motor to a support structure, and/or to secure or retain a motor within a motor mount. Latches 1211 may be used to couple a connector 1222 to an electrical contact, such as the electrical contact 111 described in reference to FIGS. 1A-1D. In some embodiments, latches 1211 may be flexible or elastically deformable. In some embodiments, latches 1211 may be rigid. Latches 1211 may be formed in conductor stamping process.

FIG. 13 shows an enlarged, partial perspective view of a motor assembly 1300. Motor assembly 1300 may include a support structure 1302. Motor assembly 1300 may include a motor 1318 retained by a motor mount 1308. Motor mount 1308 may include a surface 1366 having a shaft 1344 of motor 1318 extending therethrough. Surface 1366 may include a first electric terminal 1352 (and a second electric terminal, not shown in FIG. 13). Motor mount 1308 may include (a) linear portion(s) 1334 and (a) curved portion(s) 1336. Linear portion(s) 1334 may include a surface 1338, while curved portion(s) 1336 may include surface 1340. Connector 1322 may couple to motor mount 1308 at a first side 1310 of motor mount 1308, extend over motor mount 1308, and couple to motor mount 1308 on a second side (not shown), e.g., via a latch 1311 and a recess 1309.

Connector 1322 may include a first portion 1301, a second portion 1303, and a third portion 1305. Latches 1311 of connector 1322 may extend from a first portion 1301 of connector 1322. In some embodiments, latches 1311 may be reversibly or irreversibly coupled to recesses 1309. In some embodiments, latches 1311 may be flexible and may deflect against protrusion 1332 as connectors 1322 are inserted until latches 1311 reach recesses 1309, at which point they return to the original position. In other embodiments, latches 1311 may be rigid.

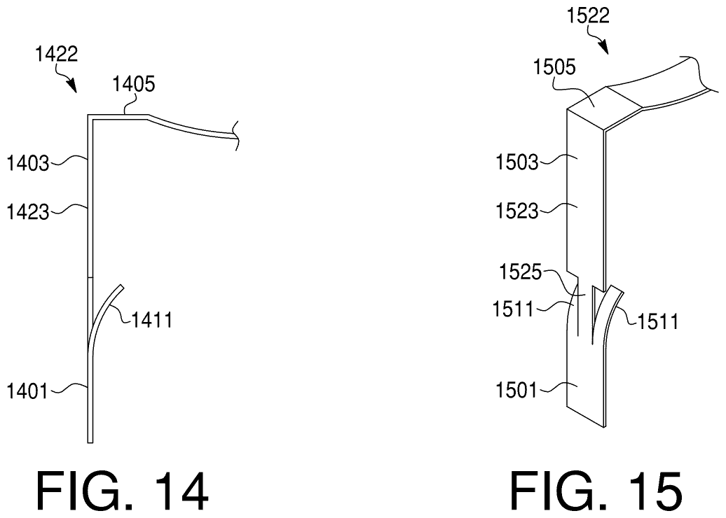

FIG. 14 shows a side view of a connector 1422, which may be similar to other connectors described herein. Connector 1422 may include a latch 1411 that extends from a connector body 1423 of connector 1422. Connector body 1423 may include a first portion 1401 and a second portion 1403. Connector 1422 may include a third portion 1405. Third portion 1405 may be substantially perpendicular to body 1423. Latch 1411 may extend into and/or couple with a recess, as described herein. In some embodiments, latch 1411 may be curved. In some embodiments, connector 1422 may electrically couple a motor to a PCB and/or a power source. In some embodiments, latches 1411 of connector 1422 are configured to couple to recesses, e.g., that are defined in a motor mount, in a motor, or in a controller.

FIG. 15 shows a perspective partial view of a connector 1522. Connector 1522 may be similar to connectors in other embodiments described herein, with the understanding that connector 1522 includes latches 1511 on both sides of an intermediate portion 1525. Connector 1522 may include a first portion 1501, a second portion 1503, and a third portion 1505. First portion 1501 includes latches and intermediate portion 1525. In some embodiments, latches 1511 may be flexible. In some embodiments, latches 1511 may deflect against, e.g., a protrusion of a motor mount as connector 1522 is inserted between a motor and the protrusion of the motor mount until latches 1511 reach, e.g., a recess or recesses, at which point latches 1511 return to their original position. In some embodiments, latches 1511 may be rigid. In some embodiments, latches 1511 may curve. In other embodiments, latches 1511 may extend from a body 1523 of the connector 1522 at an angle relative to body 1523, e.g., 10 degrees, 20 degrees, 30 degrees, 40 degrees, 45 degrees, 50 degrees, 60 degrees, 70 degrees, 80 degrees, or 90 degrees, or at an angle ranging from about 10 degrees to about 90 degrees. In some embodiments, latches 1511 of connector 1522 are configured to couple to recesses, e.g., that are defined in a motor mount, in a motor, or in a controller.

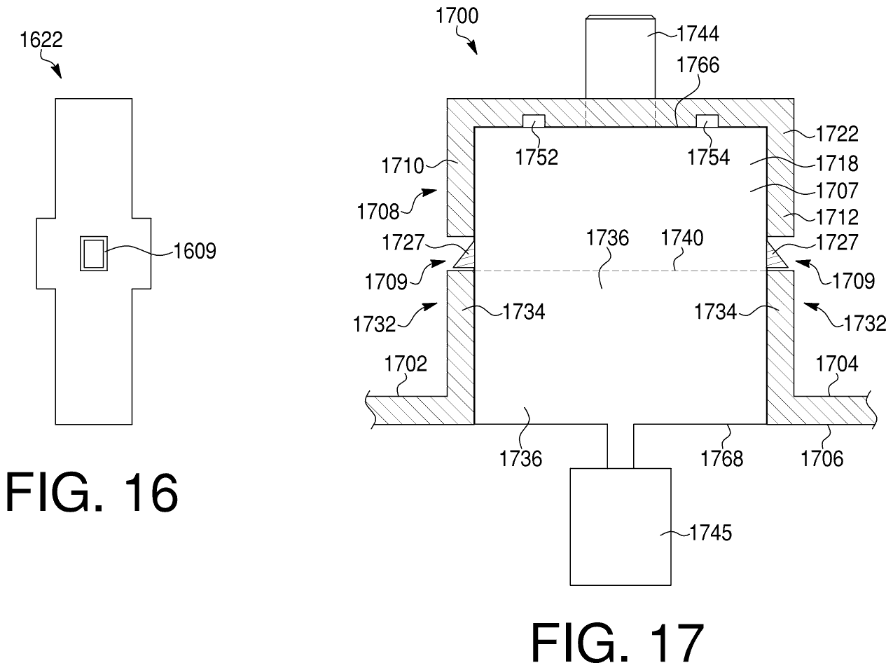

FIG. 16 shows a side view of a recess 1609 defined in a connector 1622. Connector 1622 may be similar to the other connectors described herein, and recess 1609 of connector 1622 may be configured to couple to a latch. Recess 1609 may be configured to spatially accommodate a latch to couple connector 1622, e.g., to a motor mount, a controller, or a motor.

FIG. 17 shows a partial cross-sectional view of a motor assembly 1700. Motor assembly 1700 may include a support structure 1702 including a first side 1704 opposite a second side 1706. Motor assembly 1700 may include a motor 1718, where at least a portion of motor 1718 is retained by a motor mount 1708. Motor 1718 of motor assembly 1700 may be inserted into motor assembly 1700, e.g., from second side 1706 of support structure 1702 or through a back of support structure 1702. Motor mount 1708 may have a first side 1710 and a second side 1712, and motor 1718 may include surfaces 1766, 1768 having a shaft 1744 extending therethrough, where shaft 1744 is in communication with and/or engaged to a spur gear 1745. Surface 1766 may include first and second electric terminals 1752, 1754. Motor mount 1708 may include a protrusion 1732 having one or more linear portions 1734 and one or more curved portions 1736. Linear portions 1734 may include a surface, while curved portions 1736 may include a separate surface 1740.

In some embodiments, motor assembly 1700 may include a connector 1722, which may include recess or recesses 1709 through which latches 1727 may extend to secure motor 1718 (or motor mount 1708). In some embodiments, latches 1727 may be rigid. In other embodiments, latches 1727 may be flexible. In another embodiment and as shown in FIG. 17, motor 1718 may include latches 1727 that couple with recesses defined in connector 1722.

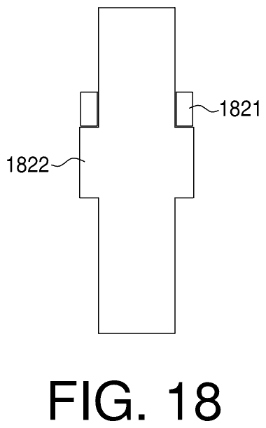

FIG. 18 shows a side view of a connector 1822 that is configured to be held in place by plastic bosses 1821 of a motor or of a motor mount. Plastic bosses 1821 may be used, e.g., to couple portions of connector 1822 to the motor or to couple portions of connector 1822 to the motor mount.

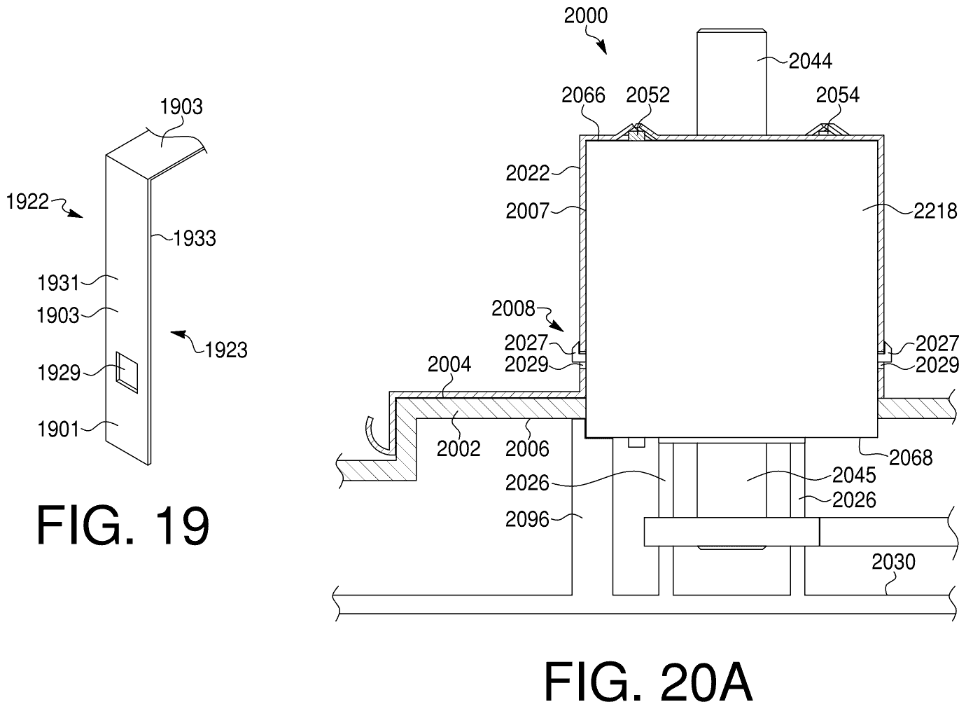

FIG. 19 shows an enlarged, perspective partial view of a connecter 1922. Connector 1922 may include a recess 1929. Connector 1922 may include a connector body 1923 including a first portion 1901 and a second portion 1903. First portion 1901 may include recess 1929. Connector 1922 may include a third portion 1903. Third portion 1903 may be substantially perpendicular to body 1923. Recess 1929 may be configured to receive or couple to a latch or other protrusion, such as those described herein. When latch is connected to recess 1929, connector 1922 may be coupled to a motor mount or to a controller, such as those described herein. In some embodiments, recess 1929 may have any appropriate cross-sectional shape, e.g., square, circular, triangular, oblong. Recess 1929 may be located at any appropriate location along connector 1922. In some embodiments, recess 1929 may be a hole or another opening extending from a first side 1931 of connector 1922 through to a second side 1933 opposite first side 1931. In some embodiments, recess 1929 may extend partially through connector 1922.

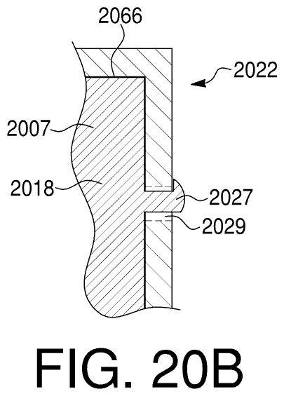

FIG. 20A shows a side view of a motor assembly 2000. Motor assembly 2000 may include a support structure 2002 including a first surface 2004 opposite a second surface 2006. Motor assembly 2000 may include a motor 2018 retained by motor mount 2008. Motor mount 2008 may include a protrusion having a side surface 2007. Motor mount 2008 may include a top surface 2066 including a first electric terminal 2052 and a second electric terminal 2054. Motor 2018, which is retained by motor mount 2008, may include a shaft 2044 extending from surface 2068 of motor 2018 and surface 2066 of motor 2018, where shaft 2044 is in communication with and/or engaged to a spur gear 2045.

Motor assembly 2000 may include a post 2096 coupled to housing 2030. Posts 2096 may structurally support motor 2018. Motor assembly 2000 may include secondary support posts 2026 that are adjacent to (and on either side of) shaft 2044. In some embodiments, latches 2027 may extend through recesses 2029 within a connector 2022 that extends over motor 2018, thereby securing connector 2022 to motor 2018. In embodiments, latches 2027 may extend through recesses 2029 within a connector that extends over motor mount 2008, thereby securing motor mount 2008 to support structure 2002. In some embodiments, latches 2027 may be any appropriate shape, e.g., a hook.

FIG. 20B shows an enlarged, partial side view of connector 2022 of motor assembly 2000. As shown in the figure, latch 2027 may couple to recess 2029 of connector 2022. In some embodiments, connector 2022 may form an electrical connection between a controller and a motor.

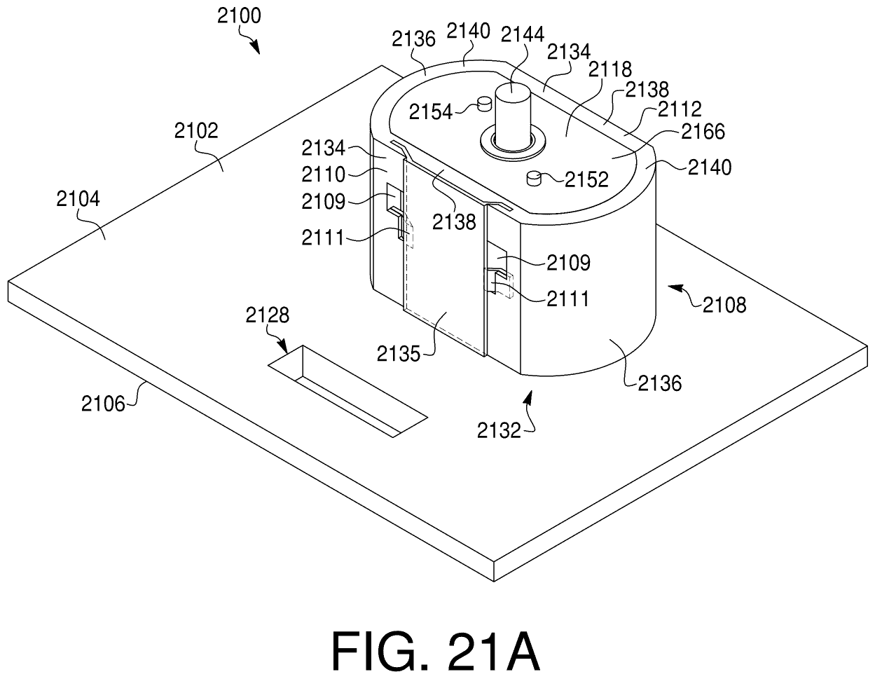

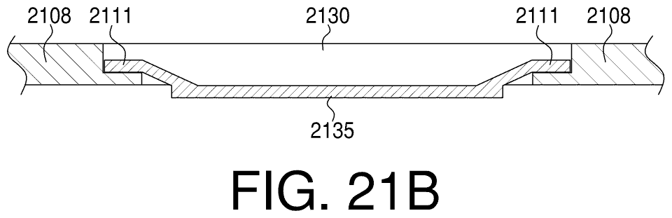

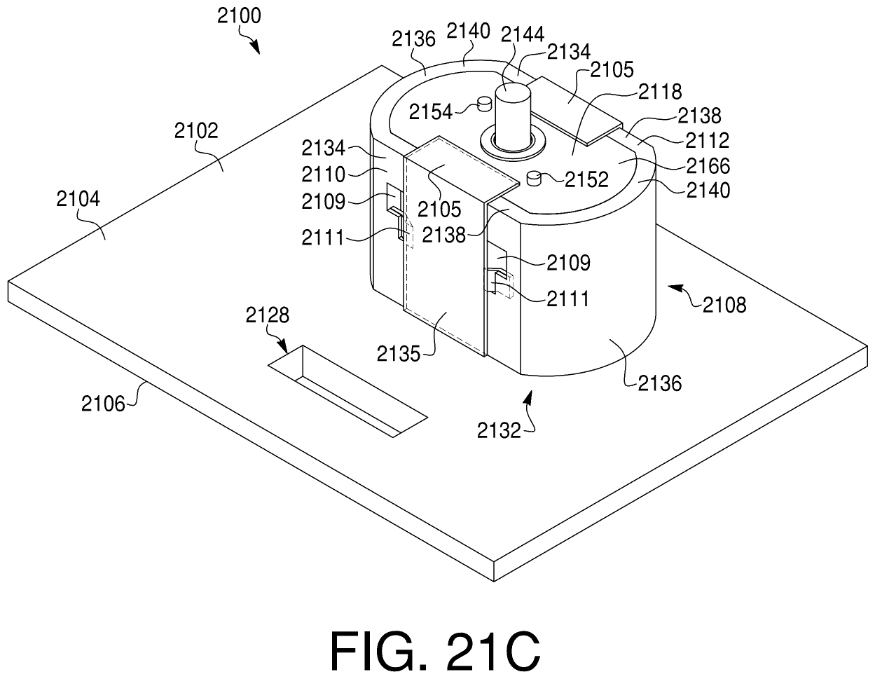

FIG. 21A-21C shows a motor assembly 2100. Motor assembly 2100 may include support structure 2102 including a first side 2104 opposite a second side 2106. Support structure 2102 may include opening 2128 through support structure 2102, which may be similar to the other opening described herein. Motor assembly 2100 may include motor mount 2108 on first side 2104 of support structure 2102. Motor mount 2108 may include a first side 2110 opposite a second side 2112. Motor mount 2108 may include a protrusion 2132 including (a) linear portion(s) 2134 and (a) curved portion(s) 2136, where protrusion 2132 extends from first side 2104 of support structure 2102. Linear portion(s) 2134 may include a surface 2138 and curved portion(s) 2136 may include a surface 2140.

Motor assembly 2100 may include motor 2118 retained by motor mount 2108. Motor 2118 may include a surface 2166 with first and second electric terminals 2152 and 2154 thereon. Motor 2118 may include a shaft 2144. In an embodiment shown in FIG. 21B, plate 2135 may be slid into or onto motor mount 2108. In some embodiments, plate 2135 may include latches 2111 thereon that engage with recesses 2109. Latches 2111 may couple to a recess 2109 or recesses 2109 of motor mount 2108. In some embodiments, plate 2135 may be configured to create a tight fit for motor 2118 in motor mount 2108. Plate 2135 may have any appropriate shape or cross-sectional area, e.g., square, rectangular, etc. While not shown in FIG. 21C, in some embodiments, portions 2105 of plate 2135 may be in direct physical contact with first and/or second electric terminals 2152, 2154.

In some embodiments, and as shown in FIG. 21B, there may be a gap 2130 between plate 2135 and motor 2118.

In some embodiments, and as shown in FIG. 21C, plate 2135 may include a portion 2105 over surface 2166. In some embodiments, plate 2135 may include electric terminals that may electrically connect motor 2118 to a power source, or that place motor 2118 in electrical communication with a controller. In some embodiments, plate 2135 may include latches (not shown) on an interior surface that may engage with motor 2118 to secure motor 2118 within motor mount 2108. Motor assembly 2100 may include one, two, three, four, or more plates 2135.

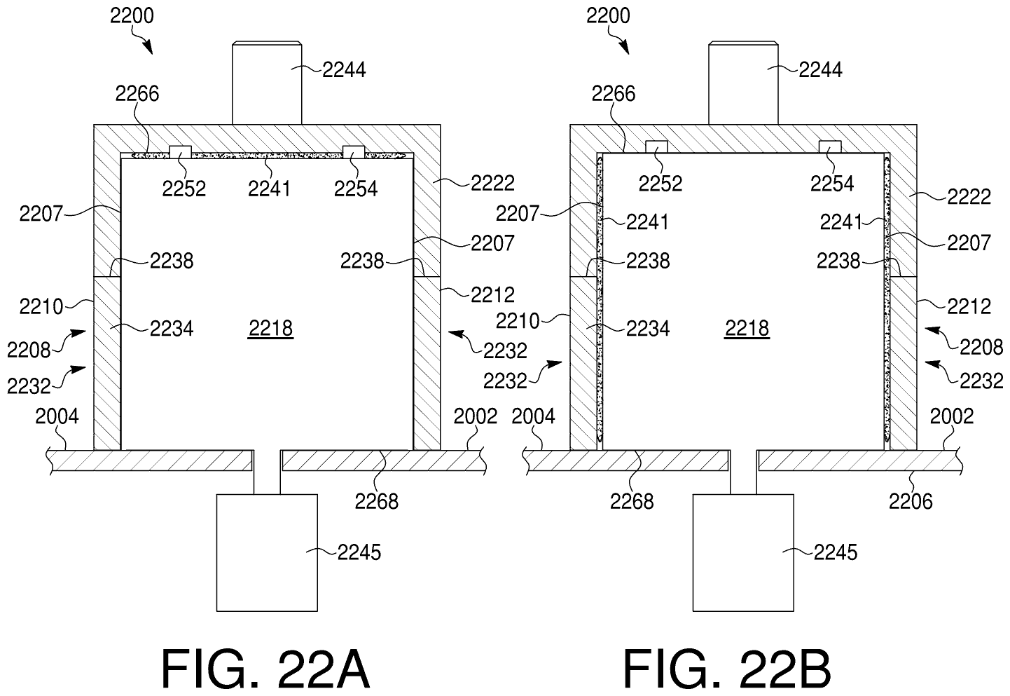

FIGS. 22A and 22B show partial cross-sectional views of a motor assembly 2200. Motor assembly 2200 may include a support structure 2002 including a first side 2204 opposite a second side 2206. Motor assembly 2200 may include a motor mount 2208 connected to first side 2204 of support structure 2002, where motor mount 2208 has a first side 2210 opposite a second side 2212. Motor mount 2208 may include a protrusion 2232 retaining motor 2218. Protrusion 2232 may include (a) linear portion(s) 2234 including a surface 2238. Motor 2218 may include a first surface 2266 opposite a second surface 2268 and a shaft 2244 extending through first surface 2266 and second surface 2268, where shaft 2244 is in communication with and/or engaged to a spur gear 2245. Linear portions 2234 of protrusion 2232 may be attached to second surface 2268 of motor 2218, e.g., by snapping together, bolting, ultrasonic welding, or soldering. In some embodiments, motor 2218 may include terminals 2252 and 2254 on surface 2266. In some embodiments, shaft 2244 may partially or fully extend through support structure 2002.

Material 2241 may be positioned between a surface 2266 of motor 2218 and connector 2222, as shown in FIG. 22A. Material 2241 may be positioned between surface 2266 of motor 2218 and connector 2222 and/or between surfaces 2207 normal to surfaces 2266, 2268 of motor 2218 and connector 2222, as shown in FIG. 22B. In some embodiments, material 2241 may to adhere any part of connector 2222 to motor 2218. In some embodiments, material 2241 may adhere any part of motor 2218 and/or to motor mount 2208. Alternatively, or in addition, material 2241 may be used to reduce movement of motor 2218 when motor 2218 is retained by connector 2222 and/or motor mount 2208. Alternatively, or in addition, material 2241 may reduce or dampen vibration transfer between motor 2218 and surrounding components. Material 2241 may be any appropriate material, including adhesives, including acrylics, epoxies, cyanoacrylates, ultraviolet-curable adhesives, silicones, polyurethanes, hot melt adhesives, and pressure-sensitive adhesives; and rubbers such as nitriles, neoprene, natural rubber, foams such as polyurethane foam, silicone foam, polyethylene foam, cork, gels such as silicone or polyurethane gels, sorbothane, felt, thermoplastic elastomers, viscoelastic materials, metals such as stainless steel or aluminum, or composites such as fiber-reinforced polymers or laminated composites.

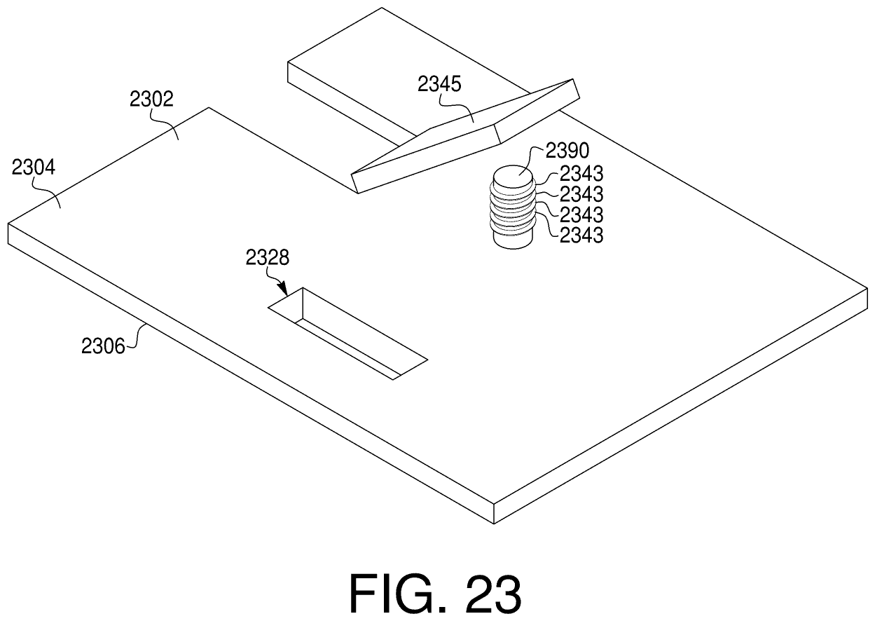

FIG. 23 shows a support structure 2302 having a first side 2304 and a second side 2306. As shown in FIG. 23, an engagement structure 2390 may be coupled to first side 2304 of support structure 2302. In some embodiments, engagement structure 2390 may secure or retain a motor to support structure 2302. Engagement structure 2390 may have any appropriate shape, e.g., substantially cylindrical, cuboidal, pyramidal, or non-polygonal. In some embodiments, engagement structure 2390 may be threaded and may thus include threads 2343. Threads 2343 may be configured for engagement with a recess of a motor mount. A motor (e.g., any motor described herein) may be retained by engagement structure 2390. In some embodiments, a motor, a shaft of a motor, or a portion of a motor mount may couple to threads 2343 of engagement structure 2390. In some embodiments, support structure 2302 may include an opening 2328. In some embodiments, a connector may extend from the motor retained by engagement structure 2390, through opening 2328, and to a controller, power source, or other component of a motor assembly. Support structure 2302 may include a conductor 2345. In some embodiments, conductor 2345 may electrically connect a motor to a power source or may communicatively connect a motor to a controller (e.g., a printed circuit board). In some embodiments, conductor 2345 may have any appropriate shape, e.g., cylindrical, cuboidal, pyramidal, non-polygonal.

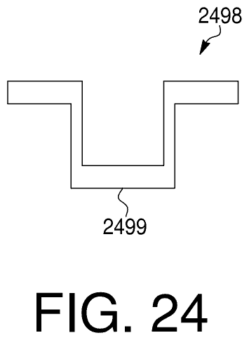

FIG. 24 shows a side partial view of a flexible member 2498 that may be utilized as part of a stamped interface fit for a motor assembly. The flexible member is configured to come into contact with a post of a support structure, e.g. to facilitate attachment of a motor or of a motor mount to a support structure. Flexible member 2498 includes a bottom portion 2499 that is configured to flex (e.g., to bend slightly) when flexible member 2498 comes into contact with a support post of a motor assembly.

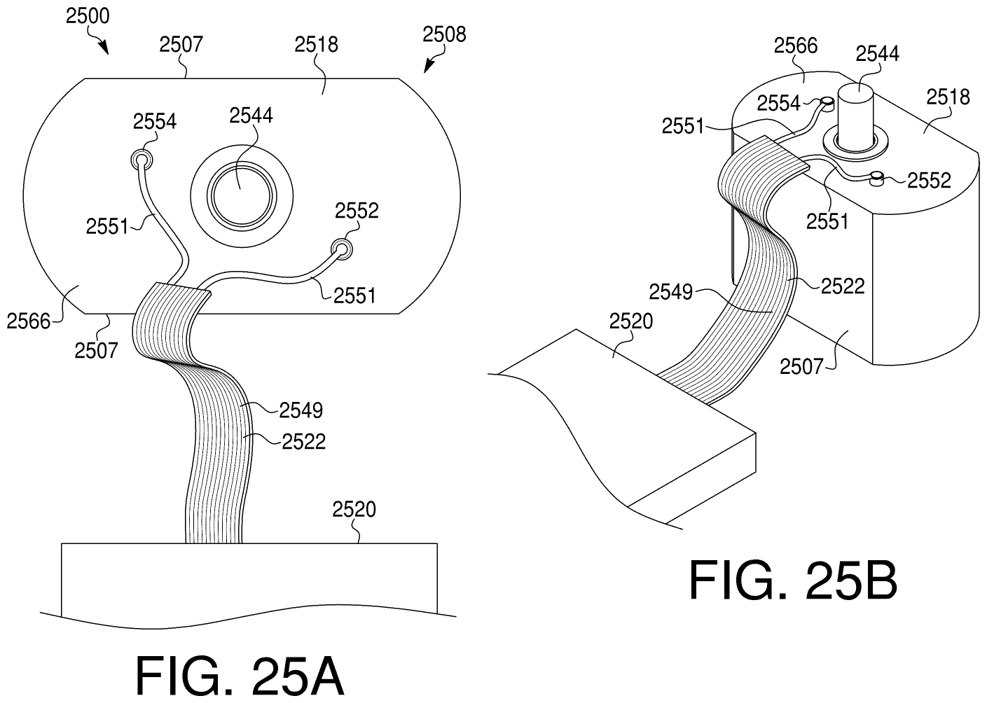

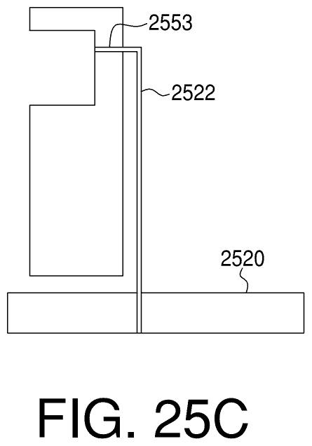

FIG. 25A-25C show various views of a motor assembly 2500 similar to other motor assemblies described herein.

As shown in FIG. 25A and FIG. 25B, motor assembly 2500 may include a motor 2518 that is retained by a motor mount, where motor 2518 includes a shaft 2544. Motor 2518 may include a top surface 2566 and side surfaces 2507. Side surfaces 2507 may be substantially perpendicular to top surface 2566.

Motor assembly 2500 further includes a connector 2522 that couples motor 2518 to controller 2520. In some embodiments, connector 2522 may be connected to a terminal 2552 or a terminal 2554 of motor 2518. In some embodiments, connector 2522 may include a first portion 2549 connected to controller 2520 and a second portion 2551 connected to a terminal 2552 or 2554 of motor 2518. In some embodiments, either or both of first portion 2549 and second portion 2551 may be flexible. In other embodiments, one of first portion 2549 or second portion 2551 of connector 2522 may be rigid. Connector 2522 may couple motor 2518 to other components apart from controller 2520, such as frequency converters, encoders, temperature sensors, pressure sensors, human-machine-interfaces, injection control units, hydraulic control systems, cooling systems, or data acquisition and telemetry systems.

FIG. 25C shows a side view of motor assembly 2500, with components of motor 2518 being omitted for ease of illustration. As shown in FIG. 25C, connector 2522 extends through an opening 2553 in a motor mount 2508. In some embodiments, connector 2522 may (indirectly) couple controller 2520 to motor 2518.

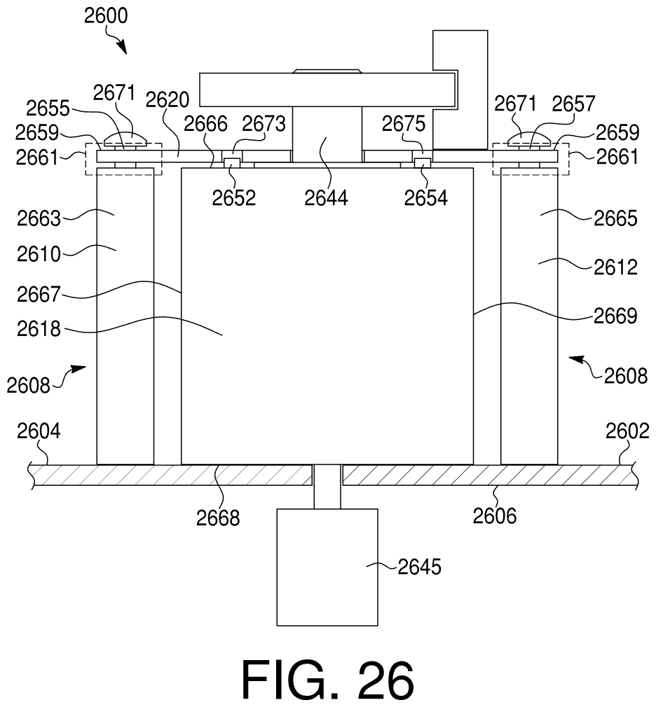

FIG. 26 shows a partial cross-sectional view of a motor assembly 2600. Motor assembly 2600 may include a support structure 2602 including a first side 2604 opposite a second side 2606. Motor mount 2608 of motor assembly 2600 may include a first side 2610 opposite a second side 2612. Motor 2618 may include a first surface 2666 opposite second surface 2668. Motor 2618 may include a shaft 2644 extending through first surface 2666 and second surface 2668, where shaft 2644 is in communication with a spur gear 2645. As mentioned, motor assembly 2600 includes first clamp 2655 and second clamp 2657. First clamp 2655 may secure controller 2620 to surface 2666 of motor 2618 at a first side 2659 of controller 2620. In some embodiments, motor 2618 and controller 2620 may be in electrical communication when controller 2620 is secured to motor mount 2608 by first clamp 2655 and/or second clamp 2657. In some embodiments, first clamp 2655 and/or second clamp 2657 may include, as examples, one or more heat stakes and/or one or more ultrasonic stakes.

Motor 2618 may include a first electrical terminal 2652 and a second electrical terminal 2654, each of which may be positioned on surface 2666 of motor 2618. Motor mount 2608 may further include a first protrusion 2663 and a second protrusion 2665 that extend from first side 2604 of support structure 2602. First and second clamps 2655, 2657 may at least partially attach controller 2620 to motor mount 2608, e.g., at protrusions 2663, 2665. In some embodiments, first clamp 2655 on first protrusion 2663 may extend from first side 2604 of support structure 2602 and secure a first side 2667 of motor 2618. In some embodiments, second clamp 2657 on second protrusion 2665 may extend from first side 2604 of support structure 2602 and secure second side 2669 of motor 2618. In some embodiments, clamps 2655 and 2657 may each include a pair of a flexible engagement structure 2671. Flexible engagement structures 2671 may be disposed around outer edges of first side 2659 and second side 2661 of controller 2620. Controller 2620 may include a first electric terminal 2673 and a second electric terminal 2675. When controller 2620 is secured using the first clamp 2655 and/or second clamp 2657, first electric terminal 2673 of controller 2620 may contact first electric terminal 2652 of motor 2618, and second electric terminal 2675 of controller 2620 may contact second electric terminal 2654 of motor 2618.

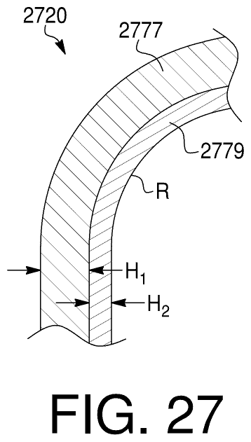

FIG. 27 shows a controller 2720 according to some embodiments. Controller 2720 may be similar to the various controllers described herein and may be flexible. In some embodiments, controller 2720 may include a first portion 2777 and a second portion 2779, which may form opposite surfaces of controller 2720. In some embodiments, first portion 2777 may include electronics, e.g., a microprocessor and other electrical components. In some embodiments, second portion 2779 may include reinforcement materials, such as fiberglass, aramid fiber, ceramic, polyimide, metal, polytetrafluoroethylene, composite materials, or glass epoxies. In some embodiments, second portion 2779 may include flexible reinforcement materials, such as polyimide films, aramid fibers, silicone rubber, flexible glass fibers, polyester films, thermoplastic elastomers, flexible metal laminates, and conductive silicone gels. In some embodiments, controller 2720 may include a radius R. In further embodiments, first portion 2777 of controller 2720 may have a first thickness H₁, and second portion 2779 of controller 2720 may include a second thickness H₂. H₁ and H₂ may be the same or different with respect to one another and may range from about 0.01 mm to about 50 mm.

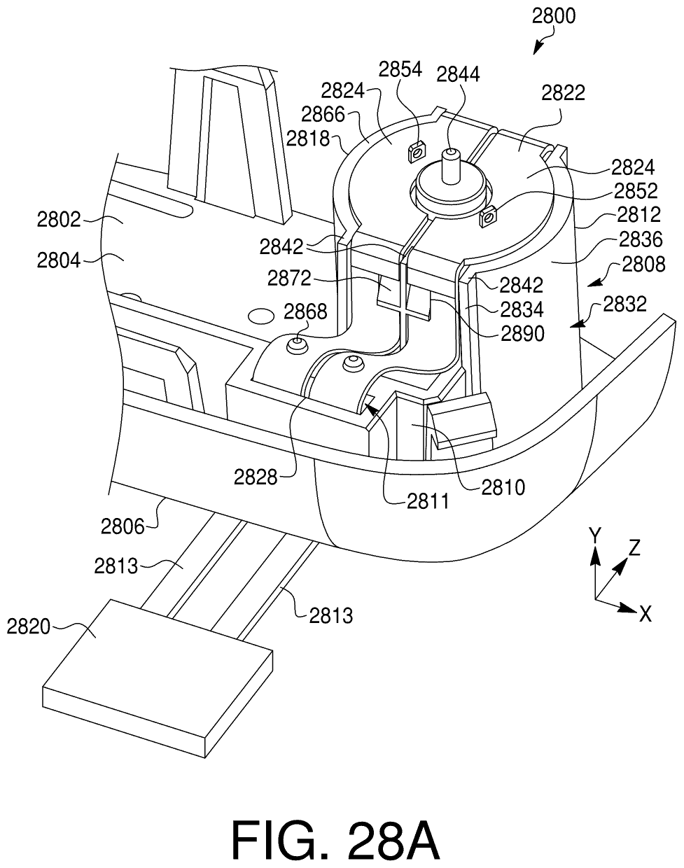

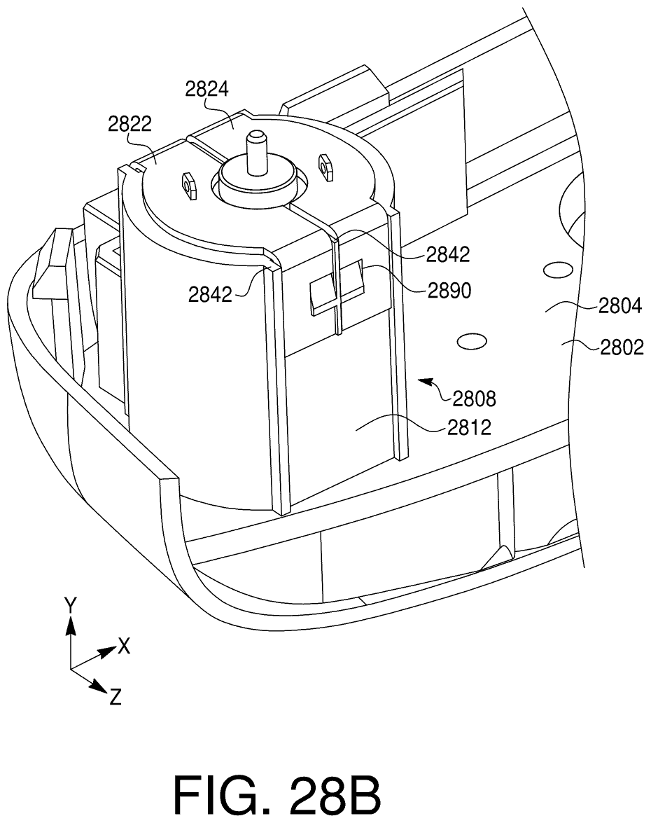

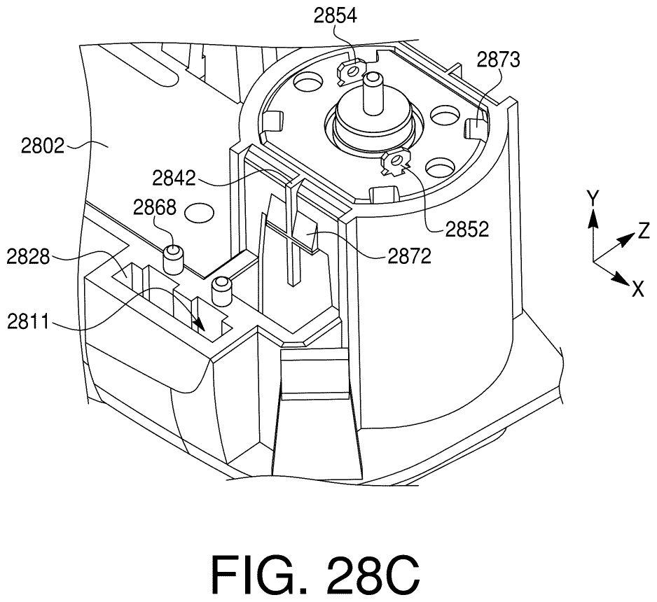

FIG. 28A shows a perspective view of a motor assembly 2800. Motor assembly 2800 may include features that aid in tri-directional, positional retention of the components of motor assembly 2800. In other words, motor assembly 2800 may facilitate positional retention of at least some of its individual components in an X direction, in a Y direction, and in a Z direction, as shown in FIG. 28A-28C. Such positional retention may be useful for maintaining the structural integrity of motor assembly 2800 throughout its lifetime and throughout a variety of uses.

Motor assembly 2800 may be used as part of a medicament delivery device, e.g., an auto-injector, as described in reference to the various motor assemblies disclosed herein.

Motor assembly 2800 may include support structure 2802. Support structure 2802 may be similar to (and may include the various materials of) the other support structures 2802 described herein. Support structure 2802 may include a first side 2804 opposite a second side 2806.

Support structure 2802 may further include a motor mount 2808. Motor mount 2808 may be similar to (and may include the same materials as) the various motor mounts described herein. As discussed in reference to the other motor mounts described herein, motor mount 2808 may be integrally formed with support structure 2802, or motor mount 2808 may be attached to support structure 2802 (e.g., through one or more of the various structures described herein or through other suitable attachment structures). Motor mount may have a first side 2810 and a second side 2812. FIGS. 28A and 28C, for example, show partial perspective views of first side 2810 of motor mount 2808, and FIG. 28B shows a partial perspective view of second side 2812 of motor mount 2808. Motor mount 2808 may include a protrusion 2832 extending vertically from first side 2804 of support structure 2802. Protrusion 2832 may include (a) linear portion(s) 2834 and/or (a) curved portion(s) 2836, as discussed in reference to the other motor mount protrusions described herein. Motor 2818 may have a top surface 2866 with a first connector 2822 and a second connector 2824 positioned thereon. At least a portion of motor 2818 may be retained by motor mount 2808.

Motor assembly 2800 further includes a controller 2820. Controller 2820 may have any suitable structure and may include any suitable materials discussed in reference to the other controllers described herein. Controller 2820 may be retained by support structure 2802, or controller 2820 may be attached to motor mount 2808, or controller 2820 may retained separately from motor mount 2808 and from support structure 2802 (e.g., in a housing separate from support structure 2802). Controller 2820 may be configured to control operation of motor 2818, e.g., speed, direction, and/or torque. In some embodiments, controller 2820 may control other functions of motor assembly 2800 or an auto-injector including motor assembly 2800, including injection speed, dose amount, dose depth, and dose temperature.

Motor assembly 2800 further includes first connector 2822 and second connector 2824. First connector 2822 and second connector 2824 may be used to place motor 2818 in electrical communication with controller 2820 and/or with a power source. First connector 2822 and second connector 2824 may be similar to (and may include the same materials as) any of the various connectors described herein. First connector 2822 and second connector 2824 may be least partially positioned over surface 2866 of motor 2818. First connector 2824 and second connector 2824 may separately extend from second side 2812 of motor mount 2808, over motor mount 2808, down first side 2810 of motor mount 2808, and into opening 2828 of support structure 2802 to (respectively) electrically couple to an electrical contact 2811 therein. Electrical contact(s) 2811 may be coupled to controller 2820 via a connection 2813, such as a flex cable, a ribbon cable, etc.

Electrical contact(s) 2811 may be any suitable material discussed in reference to the other electrical contacts described herein. Opening 2828 in support structure 2802 may be any suitable opening described herein, e.g., a slot, a gap, a hole, etc. Opening 2828 may be used to at least partially retain first and second connectors 2822, 2824 on surface 2866 of motor 2818. It is understood that while opening 2828 is shown as being rectangular in FIG. 28, opening 2828 may be any appropriate shape, e.g., square, circular, triangular, elliptical, etc.

First and second connectors 2822, 2824 may be separated from one another and at least partially retained in position by extensions 2842 that are included as part of support structure 2802 (and/or as part of motor mount 2808). As shown in FIG. 28A, in addition to extensions 2842, support structure 2802 may include studs 2868 that extend through holes and/or openings in first and second connectors 2822, 2824 (e.g., to further restrain movement of first and second connectors 2822, 2824). In some embodiments, holes and/or openings through which studs 2868, or other posts (e.g., plastic posts) included in motor assembly 2800, extend, may include features that “bite into” studs 2868. For example, holes and/or openings in first and second connectors 2822, 2824 may include barbs or flaps that deform to allow studs 2868 to extend into the holes and/or openings. The barbs or flaps may then engage with sides of studs 2868, inhibiting movement of studs 2868 out of the holes and/or openings. In some embodiments, the barbs or flaps may form an interference fit between first and second connectors 2822, 2824 and studs 2868. Studs 2868 may retain first and second connectors 2822, 2824 in position in an X or Z direction, as shown in FIG. 28A. Studs 2868 may comprise the same material as support structure 2802 or a different material, such as any suitable metal(s) or plastic(s) described herein. First and second connectors 2822, 2824 may further respectively include a cutout 2890, where each of the cutouts 2890 in the respective first and second connectors 2822, 2824 are separated by extension 2842 and enclose (an) inclined portion(s) 2872 of motor mount 2808. Inclined portion(s) 2872 of motor mount 2808 may further aid in retaining first and second connectors 2822, 2824 in position, e.g., during use of motor 2818.

Motor 2818, which is retained by motor mount 2808, includes various components, e.g., a stator, rotor, or a shaft 2844. Motor 2818 may generate rotational motional from stored energy through shaft 2844. In an embodiment, shaft 2844 may extend from a bottom surface of motor 2818 to an upper surface of motor 2818 (e.g., surface 2866). In one embodiment, when motor assembly 2800 is disposed within the housing of an auto-injector, rotation of shaft 2844 causes a needle to deploy and/or retract, and the needle may be used for injecting a medicament from a vial (in the auto-injector) into a patient.

Motor 2818 may include a first electric terminal 2852 and a second electric terminal 2854. First and second terminals 2852, 2854 may be similar to the various terminals discussed herein. First and second terminals 2852, 2854 may include any appropriate conductive material described herein, including, but not limited to, copper, brass, phosphor bronze, beryllium, nickel silver, tin plated copper, gold plated metals, silver plated metals, aluminum, stainless steel, nickel, titanium, platinum, palladium, silicides, molybdenum, and tungsten. First and second terminals 2852 and 2854 may have opposite polarities (e.g., one positive terminal 2852 or 2854 and one negative terminal 2854 or 2852).

As discussed, motor assembly 2800 may include a power source within or exterior to support structure 2802 (or within or exterior to motor mount 2808). The power source is electrically connected to the controller 2820 and/or to motor 2818.

FIG. 28B shows a rear view of motor assembly 2800. In particular, FIG. 28B shows a partial perspective view of second side 2812 of motor mount 2808 (opposite first side 2810). As shown in FIG. 28B, motor mount 2808 includes extensions 2842 that separate and enclose first connector 2822 and second connector 2824. Extensions 2842 extend vertically downward from surface 2866 of motor mount 2808 to first side 2804 of support structure 2802. Extensions 2842 may extend horizontally between first and second connectors 2822, 2824 in cutouts 2890. In embodiments, first connector 2822 and second connector 2824 are at least partially fastened to motor mount 2808 via extensions 2842 of motor mount 2808. As described, extensions 2842 of motor mount 2808 aid in retaining first connector 2822 and second connector 2824 in position, e.g., during operation of motor 2818. First connector 2822 and second connector 2824 on side 2812 of motor mount 2808 also include cutouts 2890 that enclose inclined portions 2872 of motor mount 2808. First connector 2822 and second connector 2824 may be coupled to side 2812 of motor mount 2808, e.g., using a latch (of connector 2822 or 2824) and a recess (defined in motor 2818), as discussed in reference to other motor assemblies described herein.

FIG. 28C shows another perspective view of motor assembly 2800, but with connector 2822 omitted to illustrate some of the various components of motor assembly 2800 in greater detail. For example, motor mount 2808 shown in FIG. 28C shows inclined portions 2872 that may be respectively sized, shaped, and otherwise configured to fit into cutouts 2890 of first connector 2822 and/or second connector 2824. In embodiments, inclined portions 2872 of motor mount 2808 aid in retaining first connector 2822 and/or second connector 2824 in position during operation of motor 2818. As another example, motor mount 2808 further includes fastening points 2873 that are configured to spatially accommodate portions of first connector 2822 and second connector 2824 (e.g., to attach first connector 2822 or second connector 2824 to motor mount 2808). Fastening points 2873 may be, as an example, recesses. FIG. 28C also shows extensions 2842 of motor mount (configured to separate first and second connectors 2822, 2824) and first and second electric terminals 2852, 2854 of motor 2818. Still further, FIG. 28C shows studs 2868 of support structure 2802, where studs 2868 are adjacent to opening 2828. As discussed, studs 2868 may be configured to fit into openings defined in first and second connectors 2822, 2824.

While a number of embodiments are presented herein, multiple variations on such embodiments, and combinations of elements from one or more embodiments, are possible and are contemplated to be within the scope of the present disclosure. Moreover, those skilled in the art will appreciate that the conception upon which this disclosure is based may readily be used as a basis for designing other devices, methods, and systems for carrying out the several purposes of the present disclosure.

Embodiments of the present disclosure may include the following features:
(1) A motor assembly, comprising: a support structure having a first side opposite a second side; a motor mount extending from the first side of the support structure, the motor mount including: a first side opposite a second side; and a first raised portion adjacent a second raised portion; a motor retained by the motor mount; and a first connector and a second connector mechanically securing the motor to the motor mount and configured to form an electrical connection between the motor and a controller, wherein the first connector and the second connector extend from the first side of the motor mount, over the motor mount, to the second side of the motor mount, wherein the first connector is at least partially positioned between the first raised portion and the second raised portion of the motor mount.
(2) The motor assembly of (1), wherein the motor mount includes a linear portion and a curved portion, and wherein the first connector and the second connector are at least partially positioned on the linear portion.
(3) The motor assembly of (1), wherein the motor mount further includes a third raised portion, and wherein the second connector is at least partially positioned between the second raised portion and the third raised portion of the motor mount.
(4) The motor assembly of (1), wherein the motor mount further includes: a third raised portion, wherein the second connector is at least partially positioned between the second raised portion and the third raised portion of the motor mount; a first post extending from the first side of the motor mount and disposed between the first raised portion and the second raised portion of the motor mount; and a second post extending from the first side of the motor mount and disposed between the second raised portion and the third raised portion of the motor mount, wherein the first post extends through the first connector and the second post extends through the second connector.
(5) The motor assembly of (1), wherein the first connector is configured to electrically couple a first terminal of the motor to a first terminal of a controller, and wherein the second connector is configured to electrically couple a second terminal of the motor to a second terminal of the controller.
(6) A motor assembly, comprising: a support structure including: a first side opposite a second side; an opening defined through the first side and the second side; a plurality of support posts; and a motor mount disposed adjacent to the opening, the motor mount including a protrusion extending from the first side of the support structure; and a motor retained within the motor mount, wherein the motor is retained in position by the motor mount and by the plurality of support posts.
(7) The motor assembly of (6), further comprising a controller, wherein the controller is in electrical communication with the motor.
(8) A motor assembly, comprising: a support structure having a first side opposite a second side; a motor mount; a motor retained by the motor mount, the motor including a first electric terminal and a second electric terminal; and an interlocking mechanism attaching the motor mount to the support structure.
(9) The motor assembly of (8), further comprising a controller in electrical communication with the motor.
(10) The motor assembly of (8), wherein the interlocking mechanism includes: a first projection extending from the first side of the support structure, along the first side of motor mount, and over the motor mount; and ma second projection extending from the first side of the support structure, along the second side of the motor mount, and over the motor mount to connect to the first projection.
(11) The motor assembly of (8), wherein the interlocking mechanism includes: a first projection extending from the first side of the support structure, along the first side of motor mount, and over the motor mount; and a second projection extending from the first side of the support structure, along the second side of the motor mount, and over the motor mount to connect to the first projection, wherein each of the first projection and the second projection is an elastically deforming member.
(12) The motor assembly of (8), wherein the interlocking mechanism includes: a first projection extending from the first side of the support structure, along the first side of motor mount, and over the motor mount; and a second projection extending from the first side of the support structure, along the second side of the motor mount, and over the motor mount to connect to the first projection, wherein each of the first projection and the second projection is an elastically deforming member, and wherein the motor assembly further includes a plurality of the first and second projections.
(13) A motor assembly, comprising: a support structure having a first side opposite a second side; a motor including a first electric terminal and a second electric terminal; a motor mount retaining the motor and including a first clamp and a second clamp; and a controller having a first portion opposite a second portion, wherein the controller is secured to the motor by the first clamp at the first portion of the controller, and wherein the motor and the controller are in electrical communication.
(14) The motor assembly of (13), wherein the first clamp and the second clamp each include a pair of a flexible engagement structures, wherein the first clamp at least partially encloses the first portion of the controller, and wherein the second clamp at least partially encloses the second portion of the controller.
(15) A motor assembly, comprising: a support structure having a first side opposite a second side; an opening defined in the support structure; a motor mount extending from the first side of the support structure; a motor retained by the motor mount, the motor including a first electric terminal and a second electric terminal; and a first connector and a second connector, wherein the first connector and the second connector extend into the opening defined in the support structure and are configured to form an electrical connection between the motor and a controller, and wherein the first connector and the second connector are retained in position by at least one extension of the motor mount.
(16) The motor assembly of (15), wherein the support structure includes studs adjacent to the opening in the support structure, and wherein the studs extend through holes defined in the first connector and the second connector.
(17) The motor assembly of (15), wherein the motor mount includes at least two extensions, and wherein the at least two extensions facilitate positional retention of the first connector and the second connector, or wherein the motor mount further includes inclined portions that facilitate positional retention of the first connector and the second connector.
(18) The motor assembly of (15), wherein the first connector and the second connector are spaced by an insulating material.
(19) The motor assembly of (15), wherein the motor mount includes a plurality of crush ribs positioned along inner walls of the motor mount.
(20) The motor assembly of (15), wherein the motor mount includes a plurality of studs, wherein each stud of the plurality of studs is configured to extend through one of the first connector or the second connector and form an interference fit with the one of the first connector or the second connector.

It will be apparent to those skilled in the art that various modifications and variations can be made in the disclosed motor assemblies without departing from the scope of the disclosure. Other aspects of the disclosure will be apparent to those skilled in the art from consideration of the specification and practice of the features disclosed herein. It is intended that the specification and examples be considered as exemplary only.

What is claimed is:

1. A motor assembly, comprising:
a support structure having a first side opposite a second side;
a motor mount extending from the first side of the support structure, the motor mount including:
a first side opposite a second side; and
a first raised portion adjacent a second raised portion;
a motor retained by the motor mount; and
a first connector and a second connector securing the motor to the motor mount and configured to form an electrical connection between the motor and a controller, wherein the first connector and the second connector extend from the first side of the motor mount, over the motor mount, to the second side of the motor mount, wherein the first connector is at least partially positioned between the first raised portion and the second raised portion of the motor mount.

2. The motor assembly of claim 1, wherein the motor mount includes a linear portion and a curved portion, and wherein the first connector and the second connector are at least partially positioned on the linear portion.

3. The motor assembly of claim 1, wherein the motor mount further includes a third raised portion, and wherein the second connector is at least partially positioned between the second raised portion and the third raised portion of the motor mount.

4. The motor assembly of claim 1, wherein the motor mount further includes:
a third raised portion, wherein the second connector is at least partially positioned between the second raised portion and the third raised portion of the motor mount;
a first post extending from the first side of the motor mount and disposed between the first raised portion and the second raised portion of the motor mount; and
a second post extending from the first side of the motor mount and disposed between the second raised portion and the third raised portion of the motor mount, wherein the first post extends through the first connector and the second post extends through the second connector.

5. The motor assembly of claim 1, wherein the first connector is configured to electrically couple a first terminal of the motor to a first terminal of a controller, and wherein the second connector is configured to electrically couple a second terminal of the motor to a second terminal of the controller.

6. A motor assembly, comprising:
a support structure including:
a first side opposite a second side;
an opening defined through the first side and the second side;
a plurality of support posts; and
a motor mount disposed adjacent to the opening, the motor mount including a protrusion extending from the first side of the support structure; and
a motor retained within the motor mount, wherein the motor is retained in position by the motor mount and by the plurality of support posts.

7. The motor assembly of claim 6, further comprising a controller, wherein the controller is in electrical communication with the motor.

8. A motor assembly, comprising:
a support structure having a first side opposite a second side;
a motor mount;
a motor retained by the motor mount, the motor including a first electric terminal and a second electric terminal; and
an interlocking mechanism attaching the motor mount to the support structure.

9. The motor assembly of claim 8, further comprising a controller in electrical communication with the motor.

10. The motor assembly of claim 8, wherein the interlocking mechanism includes:
a first projection extending from the first side of the support structure, along the first side of motor mount, and over the motor mount; and
a second projection extending from the first side of the support structure, along the second side of the motor mount, and over the motor mount to connect to the first projection.

11. The motor assembly of claim 8, wherein the interlocking mechanism includes:
a first projection extending from the first side of the support structure, along the first side of motor mount, and over the motor mount; and
a second projection extending from the first side of the support structure, along the second side of the motor mount, and over the motor mount to connect to the first projection, wherein each of the first projection and the second projection is an elastically deforming member.

12. The motor assembly of claim 8, wherein the interlocking mechanism includes:
a first projection extending from the first side of the support structure, along the first side of motor mount, and over the motor mount; and
a second projection extending from the first side of the support structure, along the second side of the motor mount, and over the motor mount to connect to the first projection, wherein each of the first projection and the second projection is an elastically deforming member, and wherein the motor assembly further includes a plurality of the first and second projections.

13. A motor assembly, comprising:
a support structure having a first side opposite a second side;
a motor including a first electric terminal and a second electric terminal;
a motor mount retaining the motor and including a first clamp and a second clamp; and
a controller having a first portion opposite a second portion, wherein the controller is secured to the motor by the first clamp at the first portion of the controller, and wherein the motor and the controller are in electrical communication.

14. The motor assembly of claim 13, wherein the first clamp and the second clamp each include a pair of a flexible engagement structures, wherein the first clamp at least partially encloses the first portion of the controller, and wherein the second clamp at least partially encloses the second portion of the controller.

15. A motor assembly, comprising:
a support structure having a first side opposite a second side;
an opening defined in the support structure;
a motor mount extending from the first side of the support structure;
a motor retained by the motor mount, the motor including a first electric terminal and a second electric terminal; and
a first connector and a second connector, wherein the first connector and the second connector extend into the opening defined in the support structure and are configured to form an electrical connection between the motor and a controller, and wherein the first connector and the second connector are retained in position by at least one extension of the motor mount.

16. The motor assembly of claim 15, wherein the support structure includes studs adjacent to the opening in the support structure, and wherein the studs extend through holes defined in the first connector and the second connector.

17. The motor assembly of claim 15,
wherein the motor mount includes at least two extensions, and wherein the at least two extensions facilitate positional retention of the first connector and the second connector, or
wherein the motor mount further includes inclined portions that facilitate positional retention of the first connector and the second connector.

18. The motor assembly of claim 15, wherein the first connector and the second connector are spaced by an insulating material.

19. The motor assembly of claim 15, wherein the motor mount includes a plurality of crush ribs positioned along inner walls of the motor mount.

20. The motor assembly of claim 15, wherein the motor mount includes a plurality of studs, wherein each stud of the plurality of studs is configured to extend through one of the first connector or the second connector and form an interference fit with the one of the first connector or the second connector.
